(19) United States
(12) Patent Application Publication
(10) Pub. No.: US 2012/0150202 A1
(43) Pub. Date: Jun. 14, 2012

(54) DEVICES CONTAINING A SUTURE SLEEVE AND METHODS OF MAKING AND USING

(75) Inventors: Roger Chen, Los Angeles, CA (US); John Michael Barker, Ventura, CA (US); Kenny Kinyen Chinn, Beaverton, OR (US); Ariadne Genevieve Smith, Cambridge, MA (US)

(73) Assignee: Boston Scientific Neuromodulation Corporation, Valencia, CA (US)

(21) Appl. No.: 13/310,471

(22) Filed: Dec. 2, 2011

Related U.S. Application Data
(60) Provisional application No. 61/423,030, filed on Dec. 14, 2010.

(51) Int. Cl. A61B 17/04

(52) U.S. Cl. 606/148

(57) ABSTRACT

Implantable electrical stimulation leads, lead anchors and suture sleeves, as well as methods of making and using the leads, lead anchors, suture sleeves and electrical stimulation systems are described. The lead anchors and suture sleeves present a number of strategies for anchoring a lead within tissue of a patient.

### CROSS-REFERENCE TO RELATED APPLICATIONS

This application claims the benefit under 35 U.S.C. §119(e) of U.S. Provisional Patent Application Ser. No. 61/423,030 filed on Dec. 14, 2010, which is incorporated herein by reference.

### FIELD

The present invention is directed to implantable electrical stimulation systems and methods of making and using the systems, as well as arrangements for anchoring leads of the stimulation systems within tissue. The present invention is also directed to implantable electrical stimulation leads, lead anchors and suture sleeves, as well as methods of making and using the leads, lead anchors, suture sleeves and electrical stimulation systems including any of these components.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a lead body comprising a tubular member having a wall and defining a lumen extending through the length of the tubular member and a plurality of slots through the wall. The lead body further comprises a plurality of scales and a stylet configured and arranged for insertion in the at least one lumen of the tubular member. A portion of each scale is disposed in one of the plurality of slots and through the tubular member. Each scale comprises an internal portion that extends from the respective slot into the lumen of the tubular member and an external portion that extends from the respective slot and external to the tubular member. At least a distal tip of each internal portion is configured and arranged to contact at least a portion of the stylet as the stylet is advanced through the lumen. The external portion of at least one scale is configured and arranged to be substantially flush with the external surface of a wall of the tubular member when the stylet is advanced past the internal portion of the at least one scale.

Another embodiment is a suture sleeve comprising a sleeve member having at least one tab and at least one corresponding recess. The suture sleeve further comprises a plurality of sutures coupled to the sleeve member. The sleeve member is configured and arranged to be in a first position and a second position. In the first position the sleeve member is flat. In the second position, the at least one tab and the at least one corresponding recess are coupled such that the sleeve member defines at least one lumen extending through the length of the sleeve member. The lumen is configured and arranged to receive a portion of a lead body.

Yet another embodiment is a suture sleeve comprising a tubular body defining a lumen extending through the length of the tubular body for receiving at least a portion of a lead. The tubular body further defines a first flap aperture that extends from an exterior of the tubular body through the tubular body to the lumen defined by the tubular body. The suture sleeve further comprises a first flap disposed in the first flap aperture and configured and arranged to be rotated between a first position and a second position. In the first position a first end and a second end of the first flap are disposed in the first flap aperture. In the second position the first end of the first flap is disposed in the lumen of the tubular body, and configured and arranged to contact any lead disposed in the lumen and the second end of the first flap extends beyond an exterior of the tubular body and is configured and arranged to contact adjacent tissue when the suture sleeve is implanted in tissue.

A further embodiment is a suture sleeve comprising a first portion having one or more locking tabs and at least one surface defining a first channel. The suture sleeve further comprises a second portion having one or more locking receptacles and at least one surface defining a second channel. At least a portion of the one or more locking tabs are disposed within the one or more locking receptacles to couple the first portion and the second portion together such that the first channel and the second channel form a lumen through the length of the coupled first and second portions. At least one of the one or more locking tabs comprises a sloped edge and at least one of the one or more locking receptacles comprises a sloped edge such that the sloped edges of the at least one locking tab and the at least one locking receptacle cooperate to facilitate coupling of the first portion and the second portion. At least one locking receptacle extends from a first surface of the second portion to a second surface of the second portion so that the first surface is in contact with a surface of the first portion when the first portion and the second portion are coupled, and the second surface is on an exterior of the coupled portions when the first portion and the second portion are coupled.

Another embodiment is a lead anchor comprising a lead anchor body having a top surface, a bottom surface opposite the top surface and at least one side surface that couples the top surface to the bottom surface. The lead anchor body comprises a first portion, a second portion, and a lead anchor joint disposed between the first portion of the lead anchor body and the second portion of the lead anchor body. The lead anchor further comprises a lead channel disposed in the lead anchor body and configured and arranged to receive a portion of a lead. The lead channel is open to a top surface of the lead anchor body and forms a non-linear path through the lead anchor body. The lead anchor joint is flexible and allows the first portion of the lead anchor body to be folded over on top of the second portion of the lead anchor body.

Yet another embodiment is a lead anchor comprising: a pin, a lead anchor housing, and a lead anchor lever. The lead anchor housing has one or more housing pin holes that are configured and arranged to receive a portion of the pin, one or more housing lead holes that are configured and arranged to receive a portion of a lead, and a projecting edge comprising one or more locking barbs or one or more locking receptacles. The lead anchor lever has one or more lever pin holes that are configured and arranged to receive a portion of the pin, a lead lock tab, and one or more locking barbs or one or more locking receptacles. The lead anchor can be disposed in either an open position in which the lead anchor housing and the lead anchor lever can receive a portion of the lead and a closed position in which the lead lock tab makes contact with a portion of the lead to prevent or reduce migration of the portion of the lead within the one or more housing lead holes and the one or more locking barbs are engaged in the one or more locking receptacles.

A further embodiment is a suture sleeve comprising a plurality of tubular bodies. Each tubular body defines at least one lumen that is configured and arranged to receive a portion of a lead. The suture sleeve further comprises a coupling member that couples the plurality of tubular bodies and that is configured and arranged to that allow the tubular bodies to be manipulated during implantation and then resume their original orientation with respect to each other.

Another embodiment is a suture sleeve comprising a suture sleeve base that comprises a top surface, a bottom surface and at least one side surface that couples the top surface and the bottom surface. The suture sleeve further comprises one or more tubular bodies disposed on the suture sleeve base in a pre-determined arrangement. Each tubular body defines at least one lumen extending through the length of the tubular body and each lumen is configured and arranged to receive at least a portion of a lead.

Yet another embodiment is a suture sleeve comprising a suture sleeve base having a top surface, a bottom surface opposite the top surface and at least one side surface connecting the top surface to the bottom surface. The suture sleeve base defines at least one lumen that is configured and arranged to receive a portion of a lead.

A further embodiment is a suture sleeve comprising one or more tubular bodies that define at least one lumen extending through the length of the tubular body. The at least one lumen is configured and arranged to receive a portion of a lead. The suture sleeve further comprises a plurality of locking teeth and a plurality of locking teeth connectors that couple at least one locking tooth to an exterior surface of at least one tubular body. The one or more tubular bodies are configured and arranged to be in a first position in which the locking teeth are not engaged and a second position in which two or more locking teeth are engaged to maintain the one or more tubular bodies in the second position.

Another embodiment is a suture sleeve comprising two or more tubular bodies. Each tubular body defines at least one lumen extending through the length of the tubular body. The at least one lumen is configured and arranged to receive a portion of a lead, and the two or more tubular bodies intersect to form a cross shape.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2 is a schematic view of another embodiment of an electrical stimulation system that includes a percutaneous lead body coupled to a control module via a lead body, according to the invention;

FIG. 3A is a schematic view of one embodiment of a plurality of connector assemblies disposed in the control module of FIG. 1, the connector assemblies configured and arranged to receive the proximal portions of the lead bodies of FIG. 1, according to the invention;

FIG. 3B is a schematic view of one embodiment of a connector assembly disposed in the control module of FIG. 2, the connector assembly configured and arranged to receive the proximal portion of one of the lead body of FIG. 2, according to the invention;

FIG. 3C is a schematic view of one embodiment of a proximal portion of the lead body of FIG. 2, a lead extension, and the control module of FIG. 2, the lead extension configured and arranged to couple the lead body to the control module, according to the invention;

FIG. 4A is a schematic perspective view of one embodiment of a portion of a lead body with scales and a stylet disposed in a lumen of the lead body, according to the invention;

FIG. 4B is a schematic perspective view of the portion of the lead body of FIG. 4A with the stylet advanced, according to the invention;

FIG. 4C is a schematic perspective view of a cross-section of one embodiment of a lead body with a stylet disposed in a central lumen of the lead body, conductors disposed in lumens of the lead body, and scales, according to the invention;

FIG. 4D is a schematic perspective view of one embodiment of a lead body with scales that include a suture hole, according to the invention;

FIG. 5A is a schematic perspective view of one embodiment of a sleeve member, according to the invention;

FIG. 5B is a schematic perspective view of the suture sleeve of FIG. 5A wrapped to form a tubular member, according to the invention;

FIG. 5C is a schematic perspective view of a second embodiment of a suture sleeve that includes a sleeve member, according to the invention;

FIG. 5D is a schematic perspective view of a third embodiment of a suture sleeve that includes a sleeve member, according to the invention;

FIG. 5E is a schematic perspective view of a fourth embodiment of a suture sleeve that includes a sleeve member, according to the invention;

FIG. 6A is a schematic perspective view of one embodiment a lead anchor with two detachable portions, according to the invention;

FIG. 6B is a schematic perspective view of the lead anchor of FIG. 6A with the two portions nearly attached, according to the invention;

FIG. 6C is a schematic perspective view of the lead anchor of FIG. 6A with the two portions attached and an outer sleeve, according to the invention;

FIG. 6D is a schematic cross-sectional view of one embodiment of a locking tab and a locking receptacle, according to the invention;

FIG. 6E is a schematic cross-sectional view of the locking tab engaged with the locking receptacle of FIG. 6D, according to the invention;

FIG. 7A is a schematic cross-sectional view of one embodiment of a suture sleeve that includes expanding material, according to the invention;

FIG. 7B is a schematic cross-sectional view of the suture sleeve of FIG. 7A with the expanding material at least partially expanded, according to the invention;

FIG. 7C is a schematic cross-sectional view of the suture sleeve of FIG. 7A with a portion of a lead disposed therein, according to the invention;

FIG. 7D is a schematic cross-sectional view of the suture sleeve and lead of FIG. 7C with the expanding material at least partially expanded, according to the invention;

FIG. 7E is a cross-sectional view of another embodiment of a suture sleeve that includes expanding material, according to the invention;

FIG. 7F is a cross-sectional view of the suture sleeve of FIG. 7E with the expanding material at least partially expanded, according to the invention;

FIG. 8A is a schematic perspective view of one embodiment of a lead anchor in a first open position, according to the invention;

FIG. 8B is a schematic perspective view of the lead anchor of FIG. 8A in a second, closed position, according to the invention;

FIG. 9A is a schematic perspective top view of one embodiment of a lead anchor in a first, open position, according to the invention;

FIG. 9B is a schematic perspective top view of the lead anchor of FIG. 9A in a second, closed position, according to the invention;

FIG. 9C is an exploded view of a lead anchor housing, a pin and a lead anchor lever of the lead anchor of FIG. 9A, according to the invention;

FIG. 9D is a schematic perspective bottom view of the lead anchor of FIG. 9A in a first, open position, according to the invention;

FIG. 9E is a schematic perspective bottom view of the lead anchor of FIG. 9A in a second, closed position, according to the invention;

FIG. 10A is a schematic perspective view of one embodiment of a first tubular body and a second tubular body coupled by a coupling member, according to the invention;

FIG. 10B is a schematic perspective view of an angular relationship between two tubular bodies, according to the invention;

FIG. 10C is a schematic top view of one embodiment of a suture sleeve that includes tubular bodies disposed on a suture sleeve base, according to the invention;

FIG. 10D is a schematic top view of one embodiment of a suture sleeve that includes lumens disposed in a suture sleeve base, according to the invention;

FIG. 10E is a schematic top view of another embodiment of a suture sleeve that includes lumens disposed in a suture sleeve base, according to the invention;

FIG. 10F is a schematic perspective side view of the suture sleeve of FIG. 10E, according to the invention;

FIG. 10G is a schematic perspective view of one embodiment of a suture sleeve with overlapping lumens, according to the invention;

FIG. 10H is a schematic perspective view of another embodiment of a suture sleeve with overlapping lumens, according to the invention;

FIG. 10I is a cross-sectional view of the suture sleeve of FIG. 10G along line I-I;

FIG. 10J is a schematic top view of one embodiment of a suture sleeve with an S-shaped lumen, according to the invention;

FIG. 10K is a schematic perspective view of one embodiment of a tubular, S-shaped suture sleeve, according to the invention;

FIG. 10L is a schematic perspective view of one embodiment of a suture sleeve that includes a plurality of tubular bodies disposed on a base, according to the invention;

FIG. 10M is a schematic perspective view of another embodiment of a suture sleeve that includes a plurality of tubular bodies disposed on a base, according to the invention;

FIG. 10N is a schematic perspective view of one embodiment of a suture sleeve that includes suture sleeve base lumens, according to the invention;

FIG. 10O is a schematic perspective view of one embodiment of a suture sleeve that includes multiple lumens disposed within a suture sleeve base, according to the invention;

FIG. 10P is a schematic perspective view of one embodiment of a suture sleeve that includes multiple lumens within a suture sleeve base, adhesive injection ports and adhesive tunnels, according to the invention;

FIG. 10Q is a schematic perspective view of one embodiment of an angled suture sleeve, according to the invention;

FIG. 10R is a schematic perspective view of one embodiment of a suture sleeve that includes locking teeth and locking teeth connectors, according to the invention;

FIG. 10S is a schematic perspective view of another embodiment of a suture sleeve that includes locking teeth and locking teeth connectors, according to the invention;

FIG. 11 is a schematic overview of one embodiment of components of a stimulation system, including an electronic subassembly disposed within a control module, according to the invention;

FIG. 12A is a schematic perspective view of one embodiment of a suture sleeve that includes rotatable flaps, according to the invention;

FIG. 12B is another schematic perspective view of the suture sleeve of FIG. 12A, according to the invention;

FIG. 12C is a schematic perspective view of the suture sleeve of FIG. 12A with the flaps rotated, according to the invention;

FIG. 12D is a schematic perspective view of another embodiment of a suture sleeve that includes rotatable flaps, according to the invention; and

FIG. 13 is a schematic perspective view of one embodiment of a suture sleeve with a lumen that includes protrusions, according to the invention.

### DETAILED DESCRIPTION

The present invention is directed implantable electrical stimulation systems and methods of making and using the systems, as well as arrangements for anchoring leads of the stimulation systems within tissue. The present invention is also directed to implantable electrical stimulation leads, lead anchors and suture sleeves, as well as methods of making and using the leads, lead anchors, suture sleeves and electrical stimulation systems including any of these components.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; 7,244,150; 7,672,734; 7,761,165; 7,949,395; 7,974,706; and U.S. Patent Applications Publication Nos. 2005/0165465, 2007/0150036; 2007/0219595; and 2008/0071320, all of which are incorporated by reference.

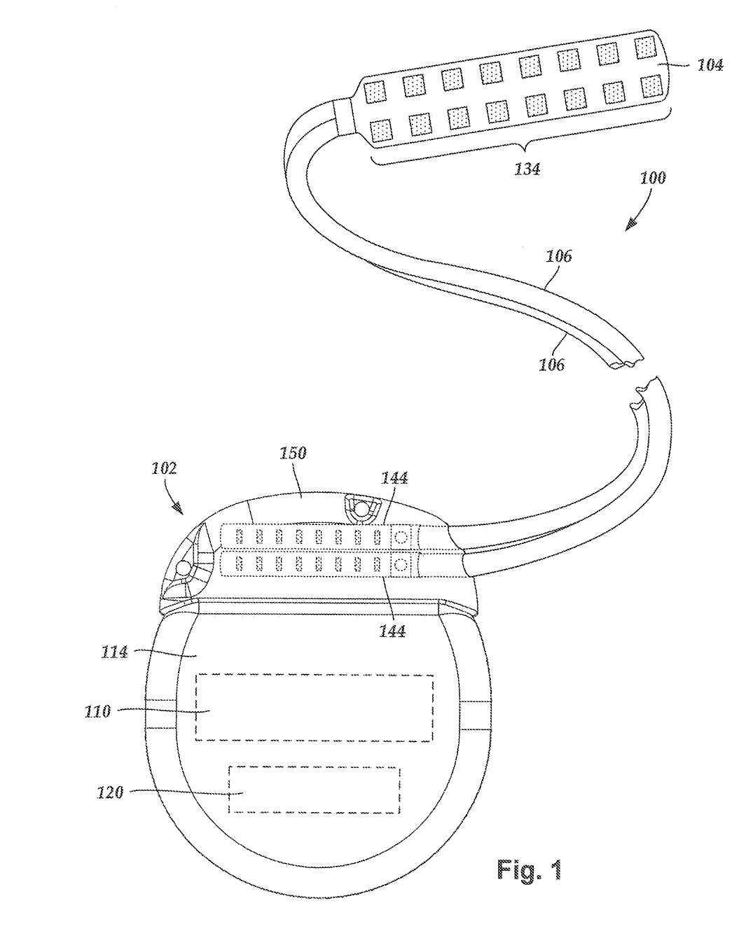

FIG. 1 illustrates schematically one embodiment of an electrical stimulation system 100. The electrical stimulation system includes a control module (e.g., a stimulator or pulse generator) 102, a paddle body 104, and one or more lead bodies 106 coupling the control module 102 to the paddle body 104. The paddle body 104 and the one or more lead bodies 106 form a lead. The paddle body 104 typically includes an array of electrodes 134. The control module 102 typically includes an electronic subassembly 110 and an optional power source 120 disposed in a sealed housing 114. In FIG. 1, two lead bodies 106 are shown coupled to the control module 102.

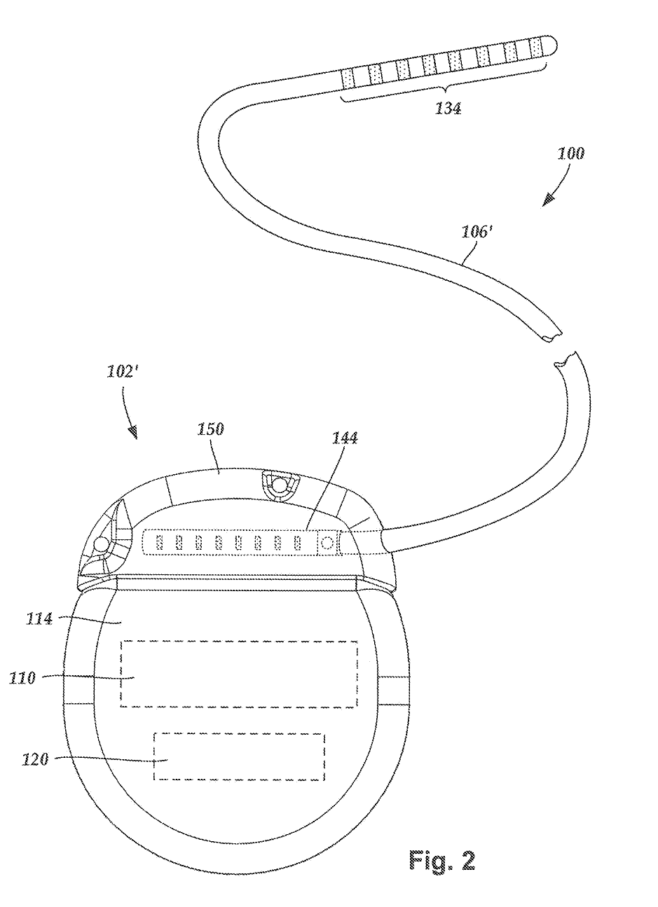

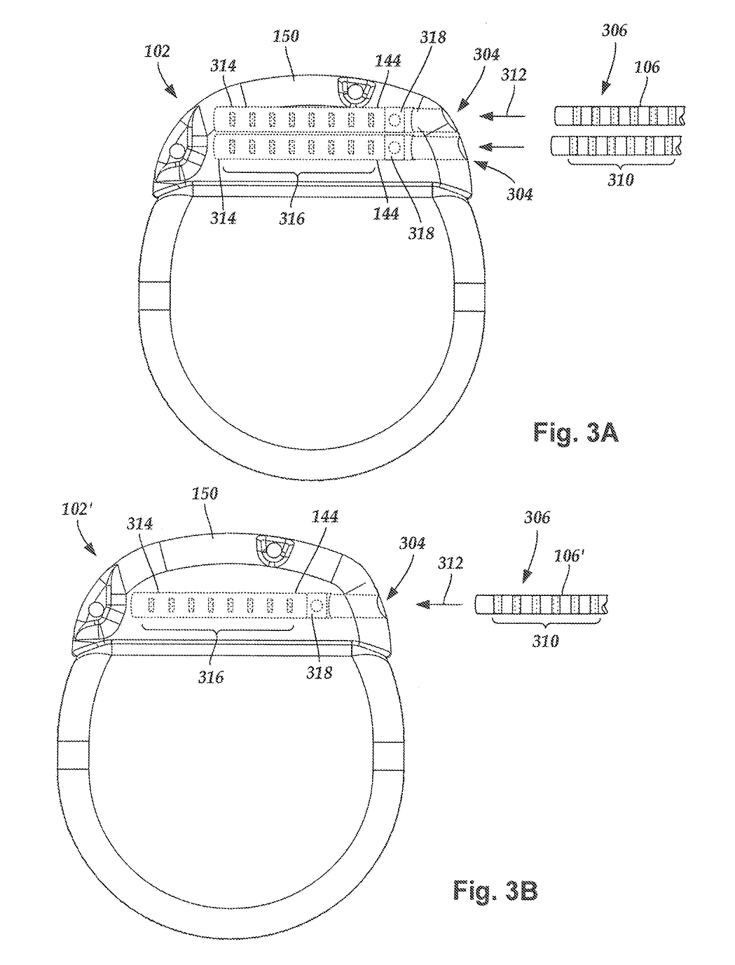

The control module 102 typically includes one or more connector assemblies 144 into which the proximal end of the one or more lead bodies 106 can be plugged to make an electrical connection via connector contacts (e.g., 316 in FIG. 3A) disposed in the connector assembly 144 and terminals (e.g., 310 in FIG. 3A) on each of the one or more lead bodies 106. The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead, as illustrated in FIG. 2. The percutaneous lead may be isodiametric along the length of the lead body 106′. The lead body 106′ can be coupled with a control module 102′ with a single connector assembly 144.

The electrical stimulation system or components of the electrical stimulation system, including one or more of the lead bodies 106, the control module 102, and, in the case of a paddle lead, the paddle body 104, are typically implanted into the body of a patient. The electrical stimulation system can be used for a variety of applications including, but not limited to, spinal cord stimulation, brain stimulation, neural stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, titanium, or titanium nitride.

The number of electrodes 134 in the array of electrodes 134 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connector assemblies (e.g., 144 in FIG. 1) disposed on, for example, the control module 102 (or to other devices, such as conductive contacts on a lead extension, an operating room cable, a lead splitter, a lead adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 3A is a schematic side view of one embodiment of a plurality of connector assemblies 144 disposed on the control module 102. In at least some embodiments, the control module 102 includes two connector assemblies 144. In at least some embodiments, the control module 102 includes four connector assemblies 144. In FIG. 3A, proximal ends 306 of the plurality of lead bodies 106 are shown configured and arranged for insertion to the control module 102. FIG. 3B is a schematic side view of one embodiment of a single connector assembly 144 disposed on the control module 102′. In FIG. 3B, the proximal end 306 of the single lead body 106′ is shown configured and arranged for insertion to the control module 102′.

In FIGS. 3A and 3B, the one or more connector assemblies 144 are disposed in the header 150. In at least some embodiments, the header 150 defines one or more ports 304 into which the proximal end(s) 306 of the one or more lead bodies 106/106′ with terminals 310 can be inserted, as shown by directional arrows 312, in order to gain access to the connector contacts disposed in the one or more connector assemblies 144.

The one or more connector assemblies 144 each include a connector housing 314 and a plurality of connector contacts 316 disposed therein. Typically, the connector housing 314 defines a port (not shown) that provides access to the plurality of connector contacts 316. In at least some embodiments, one or more of the connector assemblies 144 further includes a retaining element 318 configured and arranged to fasten the corresponding lead body 106/106′ to the connector assembly 144 when the lead body 106/106′ is inserted into the connector assembly 144 to prevent undesired detachment of the lead body 106/106′ from the connector assembly 144. For example, the retaining element 318 may include an aperture through which a fastener (e.g., a set screw, pin, or the like) may be inserted and secured against an inserted lead body 106/106′.

When the one or more lead bodies 106/106′ are inserted into the one or more ports 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connector assemblies in control modules are found in, for example, U.S. Pat. No. 7,244,150 and U.S. Patent Application Publication No. 2008/0071320, which are incorporated by reference.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector assembly 322 is disposed on a lead extension 324. The lead extension connector assembly 322 is shown disposed at a distal end 326 of the lead extension 324. The lead extension connector assembly 322 includes a contact housing 328. The contact housing 328 defines at least one port 330 into which a proximal end 306 of the lead body 106′ with terminals 310 can be inserted, as shown by directional arrow 338. The lead extension connector assembly 322 also includes a plurality of connector contacts 340. When the lead body 106′ is inserted into the port 330, the connector contacts 340 disposed in the contact housing 328 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured and arranged as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a lead extension connector assembly disposed in another lead extension. In other embodiments (as shown in FIG. 3C), the proximal end 348 of the lead extension 324 is configured and arranged for insertion into the connector assembly 144 disposed on the control module 102′.

It will be understood that the control modules 102/102′ can receive either lead bodies 106/106′ or lead extensions 324. It will also be understood that the electrical stimulation system 100 can include a plurality of lead extensions 224. For example, each of the lead bodies 106 shown in FIGS. 1 and 3A can, alternatively, be coupled to a different lead extension 224 which, in turn, are each coupled to different ports of a two-port control module, such as the control module 102 of FIGS. 1 and 3A.

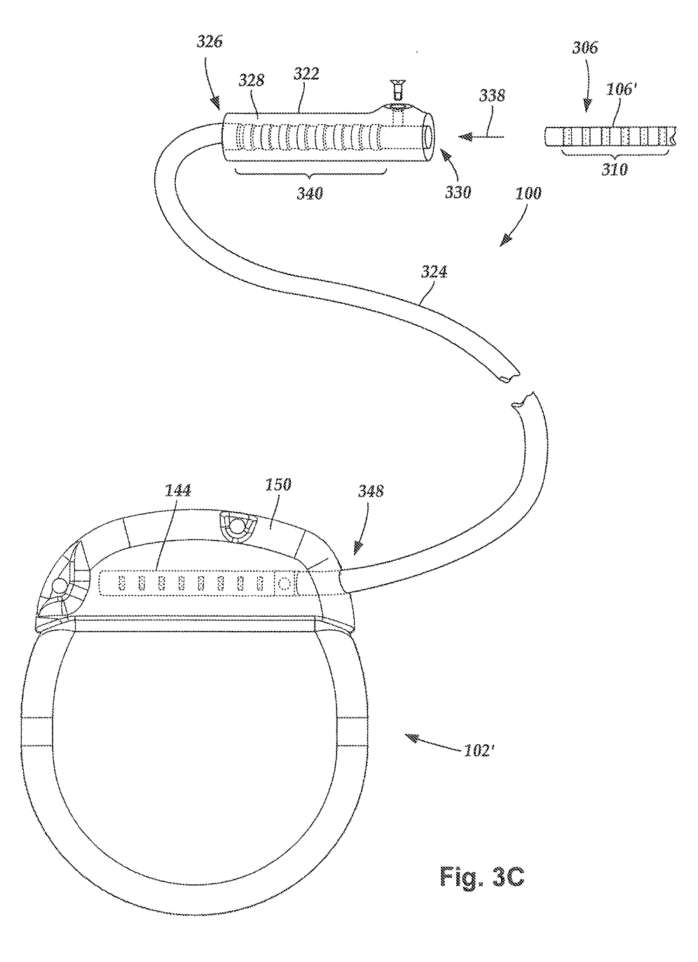

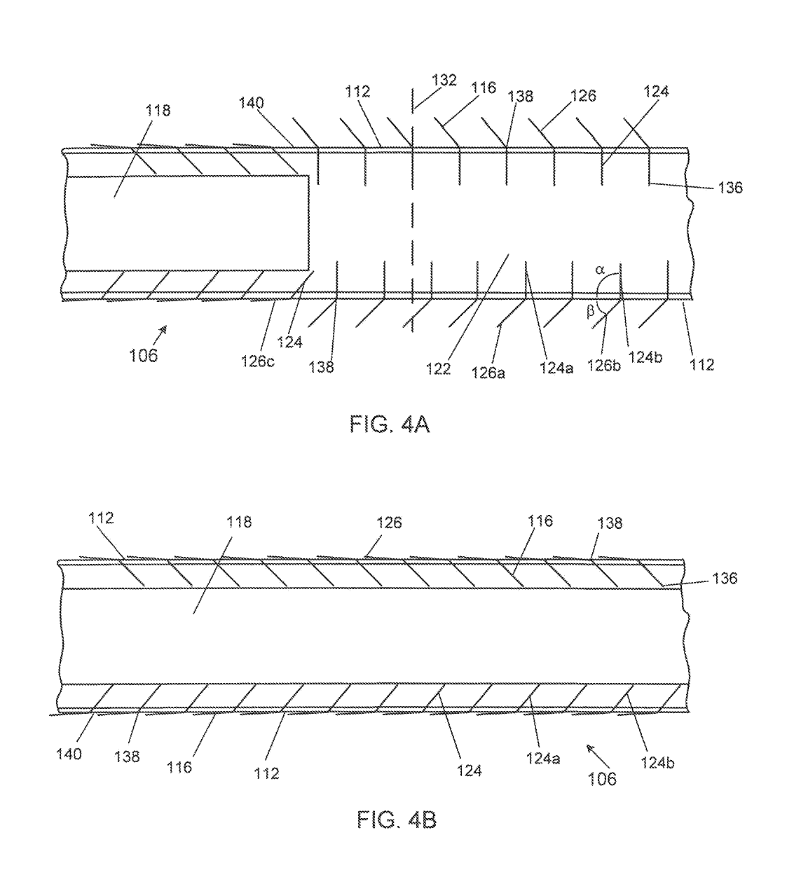

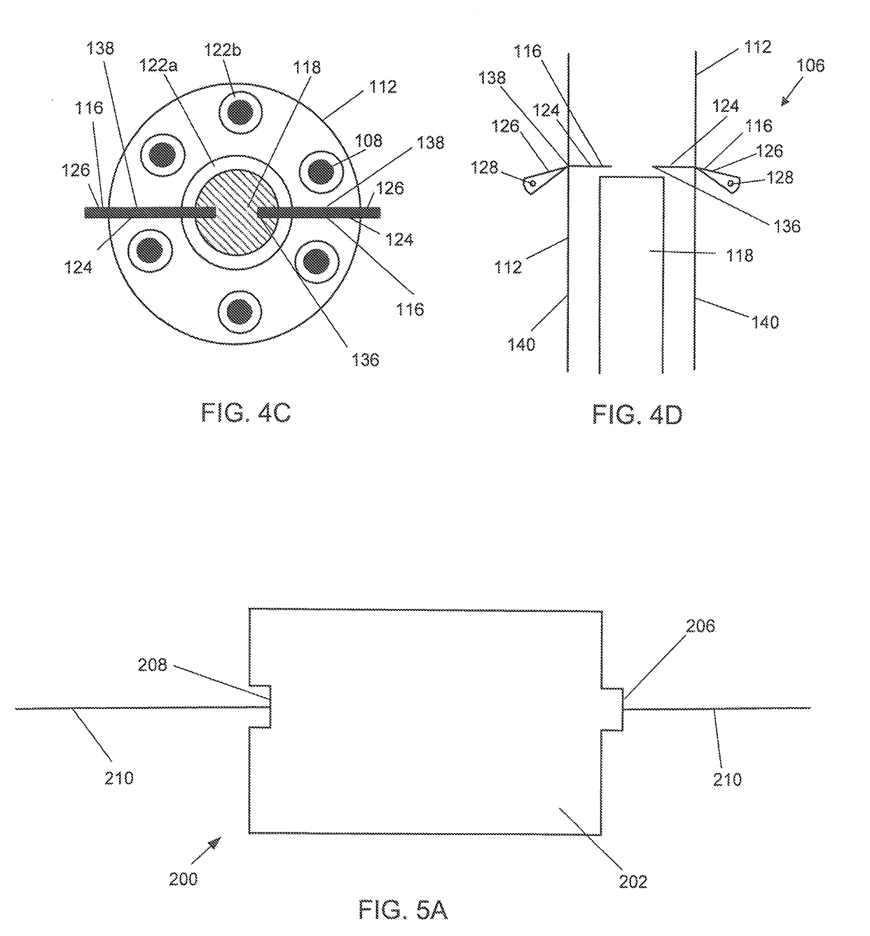

Turning to FIGS. 4A-4D, in some embodiments, a self-anchoring lead body 106 includes a tubular member 112 that defines at least one lumen 122 which extends along at least a portion the length of the tubular member 112. The at least one lumen 122 is arranged to receive a stylet 118. The at least one lumen can optionally be arranged to include one or more conductors 108 (see FIG. 4C) disposed therein for connecting electrodes on one end of the lead to contacts on the other end of the lead. As an example, the tubular member 112 defines a central lumen 122a (see FIG. 4C) and optionally defines one or more peripheral lumens 122b (see FIG. 4C). Any arrangement of the peripheral lumens and central lumen can be made. For example, the peripheral lumens 122b can be disposed around the central lumen 122a, for example, as illustrated in FIG. 4C. The central lumen 122a may be arranged to receive a stylet 118 and the peripheral lumens 122b may be arranged to hold one or more conductors 108.

The lead body 106 includes scales 116 (see FIGS. 4A-4D) that are disposed in slots 138 formed in the tubular member 112. Typically, a slot 138 in the tubular member 122 extends through the material of the tubular member 112. Any suitable arrangement of slots can be used. In some embodiments, the slots 138 are positioned in the tubular member 112 such that there is no more than one slot 138 in any one horizontal plane that is perpendicular to a longitudinal axis of a wall 140 of the tubular member 112. For example, a horizontal plane 132 that is perpendicular to a longitudinal axis 130 of a wall 140 of a tubular member 112 is illustrated in FIG. 4A. In the embodiment of FIG. 4A, the slots 138 are staggered. That is, the slots 138 in FIG. 4A are positioned in the wall 140 of the tubular member 112 such that there is no more than one slot 138 in any one horizontal plane. In some embodiments, slots 138 are disposed in a tubular member 112 such that they are arranged in a helix. It will be recognized that any other arrangement of the slots can be used including those in which slots are aligned on opposite sides of the tubular member 122.

The scales 116 include an internal portion 124 that is disposed inside the tubular member 112. The internal portion 124 of the scale 116 is at least partially disposed within a lumen 122 of the tubular member 112, for example, as illustrated in FIGS. 4A-4D. Typically, the internal portion 124 of the scale 116 extends into an interior of the tubular member 112 such that at least a distal tip 136 of the internal portion 124 is arranged to make contact with a stylet 118 as the stylet 118 is advanced past the scale 116.

In some embodiments, the internal portion 124 of at least one of the scales 116 can be positioned such that the internal portion 124 extends away from (for example, is perpendicular to or at some other angle to) the interior wall of the tubular member 112. Such an arrangement is particularly useful when the stylet 118 is not disposed within the tubular member 112. As an example, in FIG. 4A internal portions 124a and 124b are positioned such that internal portions 124a, 124b of these scales 116 are perpendicular to the wall 140 of the tubular member 112 when the stylet 118 is not advanced past these internal portions 124a, 124b. In other embodiments or in other arrangements of the present embodiment, an angle α formed by the internal portion 124 and the wall 140 of the tubular member 112 (when a stylet 118 is not present) is in the range of 30° to 110° or in the range of 50° to 100° or in the range of 60° to 100°, or the angle is at least 50°, 60°, 70°, 80°, or 85°, or the angle is 90°.

In some embodiments, advancing the stylet 118 past an internal portion 124 of a scale 116 results in the internal portion 124 being pushed in the direction of the advancing stylet 118. Typically, advancing the stylet 118 past an internal portion 124 of a scale 116 results in the internal portion 124 being pushed in the direction of the advancing stylet 118 such that the angle between the internal portion 124 and the wall 140 of the tubular member 112 is substantially increased (for example, by at least 30°, 40°, 45°, 50°, 60°, or more. For example, the angle α may be increased to at least 120°, 130°, 140°, 150°, 160°, 170°, or may even be 180°. FIG. 4B illustrates internal portions 124a and 124b after a stylet 118 has been advanced past these internal portions 124a, 124b.

The scales 116 also include an external portion 126 that extends externally from the tubular member 112 as illustrated in FIGS. 4A-4D. In some embodiments, the external portion 126 of at least one scale 116 is configured and arranged to be substantially flush with the exterior surface of the tubular member 112 when the stylet 118 is advanced past the internal portion 124 of the at least one scale 116 as illustrated in FIG. 4B. The external portion 126 of a scale 116 is “substantially flush” with the exterior surface of the tubular member 112 when an angle β (see FIG. 4A) formed by the exterior portion 126 and the wall 140 of the tubular member 112 is no more than 15°. External portion 126c illustrated in FIG. 4A is one embodiment of an external portion 126 that is substantially flush with the exterior surface of the tubular member 112.

In some embodiments, the external portion 126 of at least one scale 116 is arranged such that an angle β formed by the external portion 126 and the wall 140 of the tubular member 112 can be at least 35°, 40°, 45°, 50°, or 55° when the stylet 118 is not in contact with the internal portions 124 of the scales 116, for example when the stylet is removed from the lumen, and the scales are rotated away from the wall of the tubular member.

The internal portion 124 and external portion 126 of the scale 116 are coupled such that the angle (α+β) formed by the internal portion 124 and the external portion 126 is substantially constant. The angle (α+β) can be in the range of, for example, 90° to 145° or in the range of 100° to 135°.

In some embodiments, an external portion 126 of at least one of the scales 116 includes a suture hole 128, for example, as illustrated in FIG. 4D. The suture hole 128 can be used to anchor the lead body 106 to surrounding tissue.

In some embodiments, a method of inserting a lead body 106 includes inserting a stylet 118 into a lumen 122 of the tubular member 112 of the lead body 106 such that the stylet 118 comes into contact with at least a portion of the internal portion 124 of one or more scales 116 disposed in one or more slots 138 in the tubular member 112. The longitudinal axis of the internal portion 124 extends away from a wall 140 of the tubular member 112 before the stylet 118 comes into contact with the internal portion 124. When the stylet 118 is advanced past the internal portion 124, the internal portion 124 is pushed in the direction of the advancing stylet 118. When the stylet is advanced past the internal portion 124 of the scale, the external portion 126 of the scale 116 is moved such that the external portion 126 moves closer to, or even substantially flush with, the wall 140 of the tubular member 112.

After implantation of the lead body 106, the stylet 118 can be removed from the lumen 122 of the tubular member. Removing the stylet 118 may result in the internal portion 124 of one or more scales returning to a position in which a longitudinal axis of the internal portion 124 extends away from the wall 140 of the tubular member 112 thus anchoring the lead within the tissue of the patient. When the external portion 126 of the scale 116 is so positioned, the external portion 126 is capable of hooking into the surrounding tissue to prevent or reduce lead migration. In some embodiments, pulling back on the lead after the stylet is removed can also cause the angle between the external portion of the scales and the wall can be increased so that the scales anchor the lead body within the tissue of the patient. Optionally, the lead body can be further anchored by suturing at least one external portion 126 of one or more scales 116 to surrounding tissue using at least one suture hole 128 disposed in the at least one external portion 126.

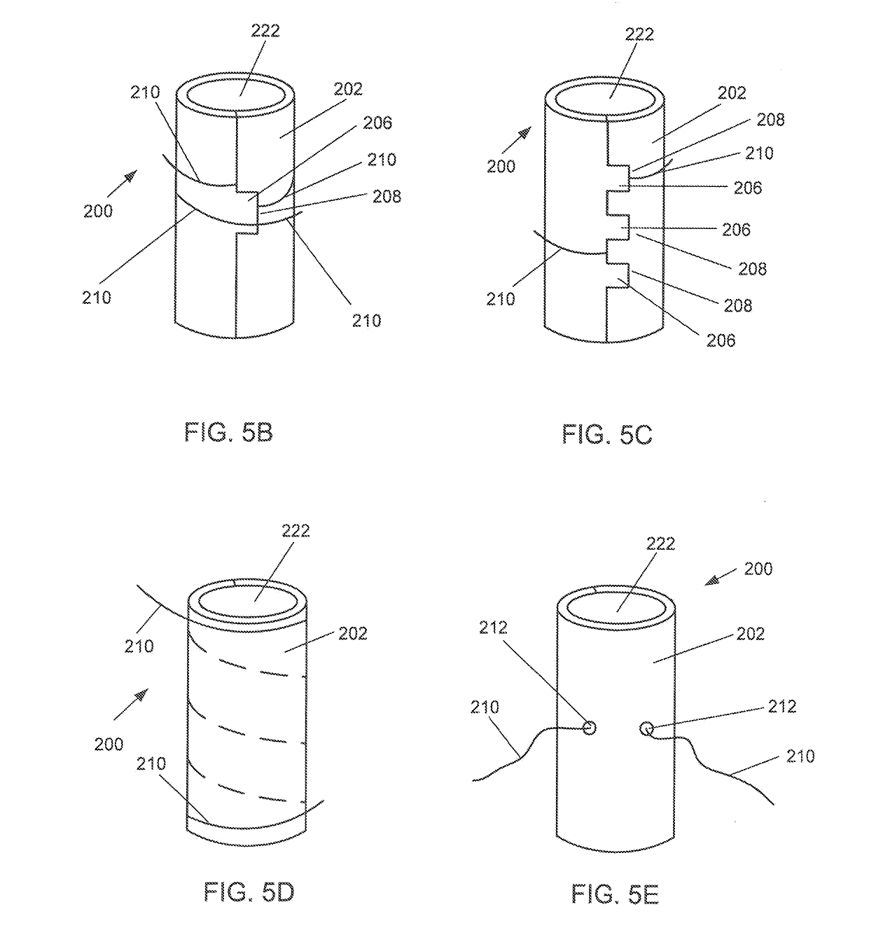

Some embodiments utilize suture sleeves for suturing a lead to tissue. Turning to FIG. 5A, in one embodiment, a suture sleeve 200 includes a sleeve member 202. The sleeve member 202 may be made of any biocompatible material including, for example, silicone, polyurethane, polyetheretherketone (“PEEK”), and the like or combinations thereof. The sleeve member 202 may be made by any process known to those of skill in the art including, for example, molding, extruding, and the like or combinations thereof.

The sleeve member 202 includes one or more tabs 206 and one or more corresponding recesses 208 as illustrated, for example, in FIG. 5A. For example, the sleeve member 202 can optionally include one or more tabs 206 on a first side of the sleeve member 202 and one or more corresponding recesses 208 on a second side of the sleeve member 202 that is opposite the first side as illustrated in FIG. 5A. In other embodiments, the tabs and recesses can be distributed on both sides of the sleeve member.

One or more sutures 210 can be coupled to the sleeve member 202. For example, FIG. 5A illustrates two sutures 210 coupled to the sleeve member 202. One or more sutures 210 may optionally be coupled to one or more tabs 206, one or more corresponding recesses 208, or a combination of tabs and recesses (or any other portions of the sleeve member 202). The sutures 210 may be coupled to the sleeve member 202 by any suitable method.

In some embodiments, the sleeve member 202 has an open position and a closed position (as well as any number of intermediate positions). In the open position, the sleeve member 202 may be laid flat, as illustrated in FIG. 5A. In the open position, the sleeve member 202 can have any shape including, for example, rectangular, square, circular, oval, or any other regular or irregular shape. In the closed position opposite sides of the sleeve member are brought together so that the sleeve member 202 defines at least one lumen 222 that extends through a length of the sleeve member 202 as illustrated in FIGS. 5B and 5C. For example, a first side of the sleeve member 202 can optionally be coupled to a second side of the sleeve member 202 that is opposite the first side such that the sleeve member 202 defines a lumen 222 extending through the length of the sleeve member 202 as illustrated in FIGS. 5B and 5C. In some embodiments, one or more tabs 206 on a first side of the sleeve member 202 are coupled with one or more corresponding recesses 208 on a second side of the sleeve member 202 that is opposite the first side such that the sleeve member 202 defines a lumen 222 extending through the length of the sleeve member 202 as illustrated in FIGS. 5B and 5C. It will be recognized that, in the closed position, embodiments of the sleeve member may have overlapping sides or sides that are positioned near each other, but not touching.

The lumen 222 defined by the sleeve member 202 when the sleeve member 202 is in the closed position can hold at least a portion of a lead body 106. As will be recognized, the lumen 222 may also receive a portion of a lead extension or other lead of a medical device. As an example, the sleeve member is wrapped around the lead during the process of moving from the open position closed position. In some embodiments, the sleeve member is formed into the closed position with the lumen and then slid over the lead.

In some embodiments, when the sleeve member 202 is in the closed position, one or more sutures 210 coupled to the sleeve member 202 are wrapped around at least a portion of the exterior of the sleeve member 202 as illustrated, for example, in FIGS. 5B, 5C and 5D. The one or more sutures 210 may additionally be coupled to the tissue of a patient. In some embodiments, wrapping one or more sutures 210 around at least a portion of the exterior of the sleeve member 202 may also secure the sleeve member 202 around a portion of the lead disposed in a lumen 222. In some embodiments, one or more sutures 210 wrapped around at least a portion of the sleeve member 202 are looped or tied to secure the sutures 210 in place.

In some embodiments, the sleeve member 202 includes one or more suture outlet holes 212. One example of suture outlet holes 212 is illustrated in FIG. 5E. A suture outlet hole 212 is to receive at least a portion of a suture 210. A suture outlet hole 212 may be disposed anywhere in the sleeve member 202. A suture 210 is threaded through suture outlet holes 212, as illustrated, for example, in FIG. 5E, to suture the sleeve member 202 in place.

When a portion of a lead is placed in a lumen 222 of the sleeve member 202 of the suture sleeve 200, the suture sleeve 200 can be used to secure the lead to surrounding tissue while reducing or preventing damage to the lead. For example, the one or more sutures 210 can be used to secure the lead to surrounding tissue. The suture(s) 210 can be wrapped around the sleeve member 202 rather than the lead itself to prevent or reduce the possibility of damage to the lead.

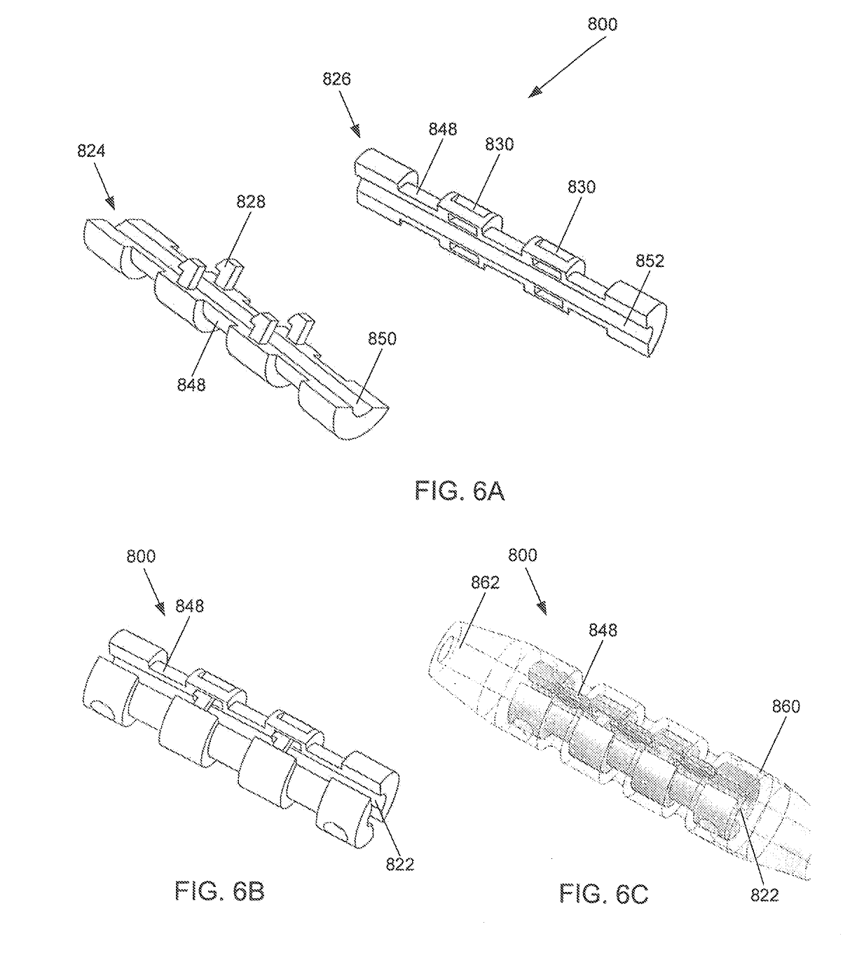

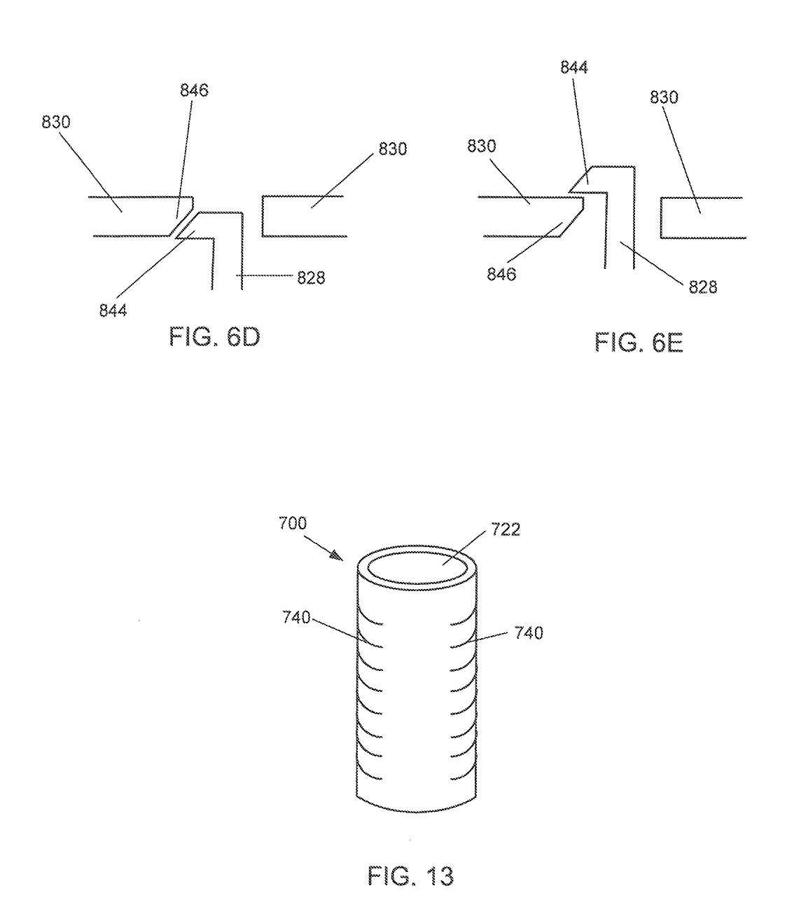

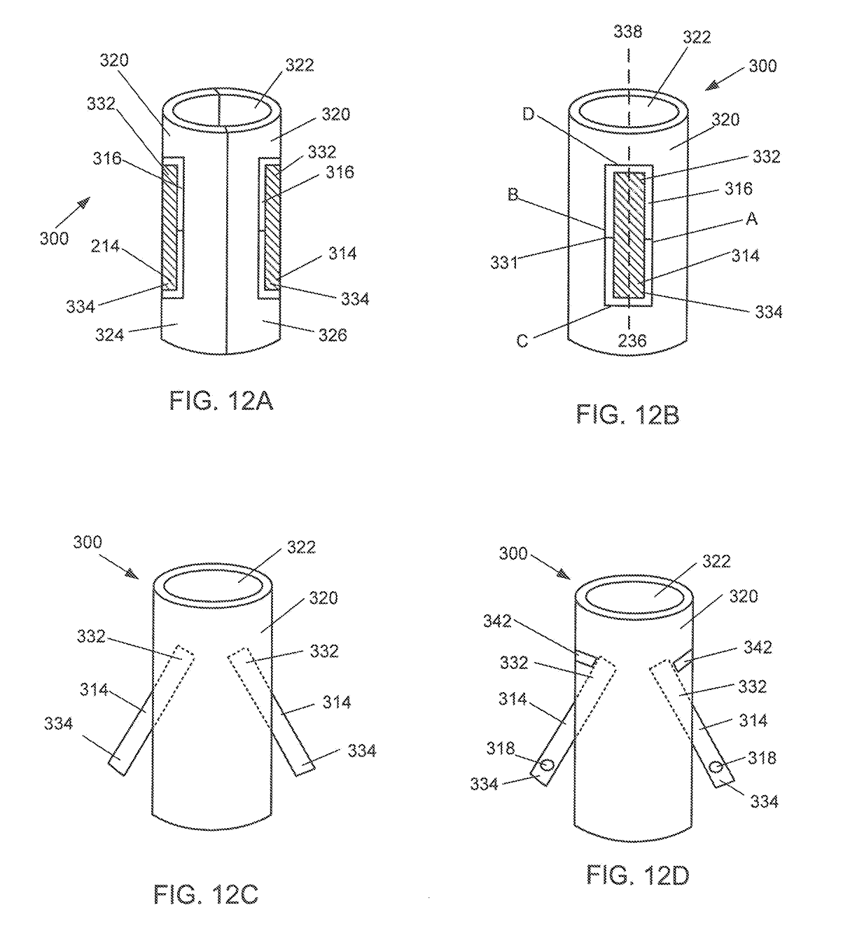

Turning to FIGS. 12A-12D, another embodiment of a suture sleeve 300 includes a tubular body 320 that defines at least one lumen 322 extending through the length of the tubular body 320 to receive at least a portion of a lead. The tubular body 320 may be formed as a single unitary member, as illustrated in FIG. 12B, or may include two or more portions that are coupled together. For example, the tubular body 320 can include a first portion 324 and a second portion 326 that may be coupled to form a tubular body 320, as illustrated, for example, in FIG. 12A. When the tubular body 320 includes two or more portions coupled together, any mechanism or arrangement for coupling the portions together can be used (including the locking tabs and corresponding locking receptacles in the arrangement described below with respect to FIGS. 6A-6E).

As illustrated in FIGS. 12A-12D, the tubular body 320 includes one or more flaps 314 that can be used to secure the lead within the tubular body. A flap 314 can be made of any biocompatible material including, for example, silicone, polyurethane, polyetheretherketone (“PEEK”), and the like or combinations thereof. A flap 314 can have any shape including, for example, a square, rectangular, circular, or oval shape or any other regular or irregular shape.

Each of the flaps 314 is situated within a flap aperture 316 formed in the tubular body, as illustrated, for example, in FIGS. 12A and 12B. In at least some embodiments, particularly if the flap aperture is square or rectangular, the flap aperture 316 is bounded, or defined, by a first pair of opposing sides (A and B) of the tubular body 320 and a second pair of opposing sides (C and D) of the tubular body 320 that connect the first pair of opposing sides, as illustrated, for example, in FIG. 12B.

In operation, the flap 314 can be rotated between at least a first position and a second position. In the first position, the flap 314 is at least partially disposed within the flap aperture 316, as illustrated, for example, in FIGS. 12A and 12B. In at least some embodiments, a majority, or even all, of the flap 314 is within the flap aperture 316 when the flap 314 is disposed in the first position.

The flap 314 is rotated about a pivot 331 to the second position in which a first end 332 is disposed within the lumen 322 of the tubular body 320 and a second end 334 is outside of the tubular body 320 as illustrated, for example, in FIGS. 12C and 12D. The pivot 331 may also be used to couple the flap 314 to the tubular body 320. The pivot may be positioned anywhere along the length of the flap 314 (i.e., the flap 314 may be symmetrically or asymmetrically divided by the pivot 331) as long as the pivot allows a portion of the flap to make contact with a lead disposed within the tubular body. In some embodiments, the second end 334 of the flap 314 that is disposed outside of the tubular body 320 can be secured to tissue of a patient, for example, by suturing the second end 334 to tissue. In at least some embodiments, one or more suture holes 318 are disposed in at least one flap 314, as illustrated, for example, in FIG. 12D. The one or more suture holes 318 may be used to suture the suture sleeve 300 to tissue of a patient.

When the flap 314 is in the first position, a lead can readily be inserted through the lumen 322 of the tubular body 320. When the flap 314 is in the second position, the first end 332 of the flap 314 contacts a portion of a lead disposed within the lumen 322 of the tubular body 320 to resist movement (e.g., longitudinal movement) of the lead within the lumen, thus anchoring the lead within the tubular body 320.

In some embodiments, the tubular body 320 includes one or more locking members 342. A locking member 342 keeps the flap 314 in the second position. One example of a locking member 342 is illustrated in FIG. 12D. In some embodiments, a locking member 342 is disposed on a surface of the tubular body 320 that faces the lumen 322 as illustrated in FIG. 12D. The locking member 342 may optionally be disposed in a locking position, in which at least a portion of the locking member 342 extends from a surface of the tubular body 320 into the lumen 322. When in the locking position, at least a portion of the locking member 342 that extends into the lumen 322 is capable of being in contact with a portion of the flap 314 such that the flap 314 is maintained in the second position. In some embodiments, the locking member 342 is disposed in a non-locking position, in which at least a portion of the locking member 342 is disposed in a locking member recess (not shown) that is at least partially or completely disposed in the tubular body 320. When in the non-locking position, the locking member 342 does not impede or prevent placement of the lead in the lumen 322 of the tubular body 320.

Turning to FIG. 13, in another embodiment of a suture sleeve, a tubular body 720 includes one or more scale-like extrusions 740 on the interior of the tubular body. The one or more extrusions 740 are provided to prevent or reduce lead migration in one direction through the lumen 722 of the tubular body 720 by catching on the exterior of the lead if the lead is advanced in the locking direction.

Turning to FIG. 6A, a suture sleeve 800 can include a first portion 824 and a second portion 826 that couple together around the lead to form the suture sleeve and lock the lead within the suture sleeve. The first portion 824 and the second portion 826 may be made of any biocompatible material including, for example, titanium, polyetheretherketone (“PEEK”), and the like or combinations thereof. In at least some embodiments, the first portion 824, the second portion 826 or both the first and second portions 824, 826 are made of a biocompatible material that is more rigid than the material of the lead.

The first portion 824 defines at least one channel 850 extending through the length of the first portion 824 for receiving the lead. Typically, at least one surface of the second portion 826 defines at least one channel 852 extending through the length of the second portion 826 for receiving the lead. When coupled together, the channel 850 of the first portion 824 and the channel 852 of the second portion 826 define a lumen 822, as illustrated, for example, in FIGS. 6B and 6C. Typically, the lumen 822 extends through the length of the coupled first and second portions 824, 826. In at least some embodiments, the channel 850 of the first portion 824, the channel 852 of the second portion 826, or both the channels 850, 852 include one or more gripping protrusions (not shown) that are provided to prevent or reduce migration of a portion of a lead disposed within the lumen 822 formed by channels 850 and 852 when the first portion 824 and the second portion 826 are coupled together around the lead. In some embodiments, the channel 852 has a slightly smaller diameter than the lead to be inserted into the channel to facilitate gripping the lead.

To facilitate coupling of the two portions, 824, 826, the first portion 824, the second portion 826, or both the first and second portions 824, 826 of a suture sleeve 800 may include one or more locking tabs 828. The first portion 824, the second portion 826, or both portions 824, 826 may include one or more corresponding locking receptacles 830. A locking receptacle 830 receives at least a portion of a locking tab 828.

A locking tab 828 or a locking receptacle 830 can be made of any biocompatible material including, for example, titanium, polyetheretherketone (“PEEK”), and the like or combinations thereof. In some embodiments, a locking tab 828 or a locking receptacle 830 is made from a material that is more rigid than the lead material. In some embodiments, one or more locking tabs 828 or locking receptacles 830 are made from a biocompatible material that is capable of bending or stretching to allow the one more locking tabs 828 to engage more easily with the one or more locking receptacles 830. The locking tabs 828 and locking receptacles 830 can be made from the same or different biocompatible material, which may be the same or different material than the first portion 824 and the second portion 826.

In some embodiments, the locking tab 828 includes at least one sloped edge 844. One example of a sloped edge 844 of a locking tab 828 is illustrated in FIGS. 6D and 6E. The sloped edge 844 of the locking tab 828 optionally facilitates engagement of the locking tab 828 into a locking receptacle 830.

The locking receptacle 830 optionally includes at least one sloped edge 846, as illustrated, for example, in FIGS. 6D and 6E. The sloped edge 846 of the locking receptacle 830 can facilitate engagement and locking of the locking tab 828 in a locking receptacle 830.

FIG. 6D illustrates one embodiment of a sloped edge 846 of a locking receptacle 830 and a corresponding sloped edge 844 of a locking tab 828 that allows the locking tab 828 to be more easily engaged and locked in the locking receptacle 830. As illustrated in FIG. 6D, the sloped edge 844 of the locking tab 828 slides over the sloped edge 846 of the locking receptacle 830 and facilitates the locking tab 828 locking in the locking receptacle 830 (see FIG. 6E). In some embodiments, the first portion 824 and the second portion 826 can be coupled together by aligning one or more locking tabs 828 on one portion with one or more locking receptacles 830 on the other portion and squeezing the portions together such that at least a portion of the one or more locking tabs 828 become disposed in the one or more locking receptacles 830.

A first portion 824, a second portion 826, or both portions 824, 826 of a suture sleeve 800 can include one or more suture grooves 848, as illustrated, for example, in FIGS. 6A and 6B. A suture groove 848 receives a portion of a suture. Typically, the suture groove 848 is recessed as compared to parts of the first or second portions 824, 826 that are adjacent to the suture groove 848. In at least some embodiments, the suture groove 848 extends around the circumference of the coupled first and second portions 824, 826 as illustrated, for example, in FIGS. 6B and 6C.

In some embodiments, the suture sleeve 800 includes an outer sleeve 860 as illustrated, for example, in FIG. 6C. The outer sleeve 860 can be disposed over at least a part of the first portion 824, the second portion 826, or both the first and second portions 824, 826. Optionally, a portion of the outer sleeve 860 can extend beyond a surface of the first portion 824, the second portion 826 or both the first and second portions 824, 826 as illustrated in FIG. 6C. The outer sleeve 860 may form a lumen 862 that is coextensive with the lumen 822 formed by the first and second portions 824, 826 as illustrated, for example, in FIG. 6C. The exterior surface of the outer sleeve 860 may define one or more suture grooves 848. One or more coating suture grooves 848 may coincide with the one or more suture grooves 848 disposed on the first portion 824, the second portion 826 or both the first and second portions 824, 826.

The outer sleeve 860 can be made of any biocompatible material including, for example, silicone, polyurethane, polyetheretherketone (“PEEK”), and the like or combinations thereof. In some embodiments, the outer sleeve 860 is made of a biocompatible, elastic material including, for example, silicone. The outer sleeve 860 may be more flexible than the first and second portions 824, 826.

Although each of the embodiments has been described separately, it will be understood that many of the elements of each of these embodiments may be included in any of the other embodiments described herein. For example, any of the embodiments may also include one or more flaps and corresponding flap apertures, one or more scale-like extrusions 228 on an interior surface, or locking tabs and receptacles.

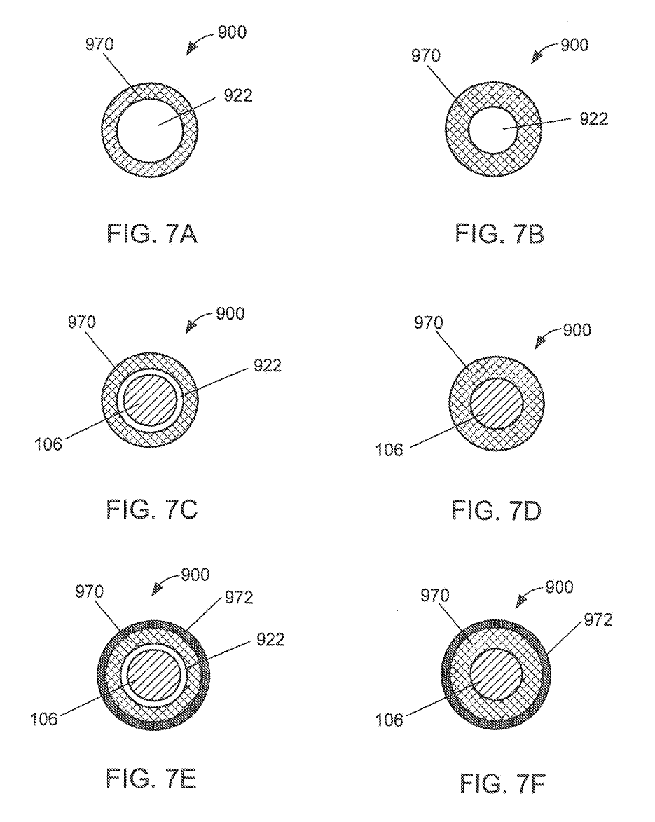

Turning to FIG. 7A, in some embodiments, a suture sleeve 900 includes an expanding material 970 that expands when it comes into contact with fluid (e.g., water, saline, blood, etc.). Any suitable material can be used including, but not limited to, hydrogels and Tecophilic™ materials. FIG. 7A illustrates a cross section of one embodiment of a suture sleeve 900 that includes expanding material 970 that has not been contacted with a fluid. FIG. 7B illustrates a cross section of the suture sleeve 900 of FIG. 7A after the suture sleeve 900 has been contacted with fluid.

The suture sleeve 900 can be made entirely of expanding material 970 or, alternatively, only part of the suture sleeve is made of expanding material 970. For example, the suture sleeve 900 may include expanding material 970 that is disposed on a surface of the lumen 922 of the suture sleeve 900. In at least some embodiments, the expanding material 970 defines the lumen 922 of a suture sleeve 900. For example, the lumens 922 of the suture sleeves of FIGS. 7A and 7B are defined by a layer of expanding material 970.

A method of implanting a lead can include inserting at least a portion of a lead, such as a portion of a lead body 106, into a lumen 922 of a suture sleeve 900 that includes expanding material 970, as illustrated, for example, in FIG. 7C. The suture sleeve 900 then contacts a fluid such as, for example, water, saline, blood, other patient fluids, and the like, and the expanding material 970 expands so that the suture sleeve 900 is more securely coupled to the lead. In some embodiments, the expanding material 970 expands such that the expanding material 970 completely fills the portion of the lumen 922 not occupied by the lead body 106 as illustrated, for example, in FIG. 7D.

Optionally, the suture sleeve 900 can be sutured or otherwise secured to a portion of a lead, such as a portion of a lead body 106. The suture sleeve 900 which has been sutured or otherwise secured to a portion of a lead may also be secured to a tissue of a patient by one or more sutures.

In some embodiments, the suture sleeve 900 also includes an external layer 972 disposed around at least a portion of the expanding material 970. The external layer 972 can be formed of any biocompatible material including, for example, silicone, polyurethane, and the like or combinations thereof, as illustrated, for example, in FIGS. 7E and 7F. The external layer 972 may prevent or reduce the expansion of the expanding material 970 in the direction of the external layer 972 such that the expanding material 970 is more likely to expand in the direction of the lumen 922.

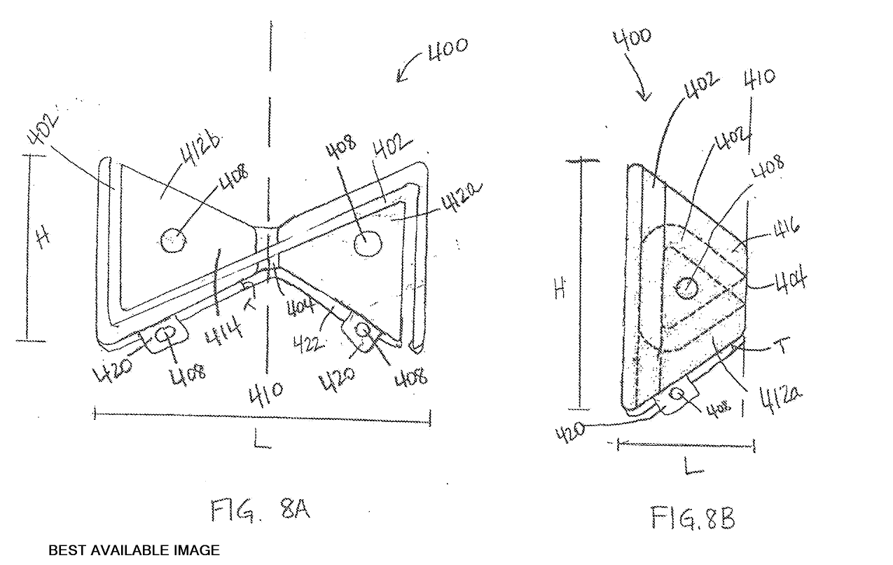

Turning to FIG. 8A, in some embodiments, a lead anchor 400 includes a lead anchor body 412. The lead anchor body 412 can be made of any biocompatible material including, for example, silicone, polyurethane, polyetheretherketone (“PEEK”), and the like or combinations thereof. The lead anchor body 412 can have any suitable shape that provides a non-linear lead channel as discussed below. For example, in one embodiment, when in the open first position, the lead anchor body 412 has a bowtie shape as illustrated in FIG. 8A.

The lead anchor body 412 includes a top surface 414 and a bottom surface 416. The lead anchor body 412 forms a lead channel 402 that is disposed in the lead anchor body 412. The lead channel 402 receives a portion of a lead body 106 such that the lead body 106 is held securely within the lead channel 402. The lead channel 402 may be open at the top surface 414, bottom surface 416, or both surfaces. Alternatively or additionally, the lead channel 402 may be closed at the top surface 414, bottom surface 416, or both surfaces. In some embodiments, the lead channel 402 accepts a portion of a lead body 106 such that the lead body 106 is flush with a top surface 414, bottom surface 416, or both surfaces when disposed in the lead channel 402.

The lead channel 402 can have any suitable non-linear shape such as, for example, an “S” or “Z” shape as illustrated in FIG. 8A. Preferably, the lead channel has one, two, three, four, or more turns where the direction of the lead channel changes. The lead channel 402 may include a full loop of the lead.

The lead channel 402 can have any suitable dimensions which may be selected based, at least in part, on the dimensions of the anticipated lead body. In some embodiments, the lead channel diameter is 1.3 mm (0.05 inches), 1.5 mm (0.06 inches), 1.8 mm (0.07 inches), 1.9 mm (0.075 inches), 2 mm (0.08 inches), or more. The lead channel 402 may include extrusions (see, e.g., the embodiment of FIG. 13 discussed above) or protrusions into the lead channel that prevent or inhibit migration of the lead within the lead channel 402.

The lead anchor body 412 may include a lead anchor joint 404, particularly if the lead anchor body is a single structure. The lead anchor joint 404 can be disposed at a midline of the lead anchor body 412 such that an axis of symmetry 410 bisects the lead anchor body 412 as illustrated, for example, in FIG. 8A. Alternatively, the lead anchor joint can be at any other suitable position along the lead anchor body. In other embodiments, the lead anchor body can be formed using separate parts (e.g. a top part and a bottom part) that fit together to hold the lead anchor.

Typically, the lead anchor body 412 is made of one or more biocompatible materials. The lead anchor joint 404 and the remainder of the lead anchor body 412 can be made from the same or different materials.

The lead anchor joint 404 is configured and arranged to be flexible such that a first portion of the lead anchor body 412a (see FIG. 8A) can be disposed over a second portion of the lead anchor body 412b, for example, as illustrated in FIG. 8B. In at least some embodiments, the lead anchor body 412 has a symmetrical shape along an axis of symmetry 410 (see FIG. 8A) such that the first portion of the lead anchor body 412a can be folded about an axis of symmetry 410 to allow the first portion of the lead anchor body 412a to be disposed over an identically or similarly shaped second portion of the lead anchor body 412b. In some embodiments, the first portion of the lead anchor body 412a (ignoring the lead channel) is a mirror image of the second portion of the lead anchor body 412b about the axis of symmetry 410.

As described above, the lead anchor 400 has both a first position and a second position (as well as intermediate positions between the first and second positions). In the first position, the first portion 412a and the second portion 412b of the lead anchor body 412 lie in the same horizontal plane and the lead channel 402 is accessible from a top surface 414 of the lead anchor body 412 as illustrated, for example, in FIG. 8A. In the second position, the first portion 412a of the lead anchor body is disposed over the second portion 412b of the lead anchor body, as illustrated, for example, in FIG. 8B, typically with the lead body held within the lead anchor.

The lead anchor body 412 can have any suitable dimensions. In some embodiments, the length L of the lead anchor body 412 when in the open first position is in the range from 0.2 to 3 cm. In some embodiments, the height H of the lead anchor body 412 in the open first position is in the range of 0.2 to 3 cm. In some embodiments, the thickness T of the lead anchor body 412 in the open first position is in the range of 1 to 10 mm or in the range of 1 to 3 mm.

The lead anchor body 412 can optionally include one or more suture holes 408, as illustrated, for example, in FIGS. 8A and 8B. A suture hole 408 can be used to suture or otherwise secure the lead anchor 400 to a tissue of a patient such as the patient facia or ligament.

In some embodiments, a suture hole 408 extends from a top surface 414 of the lead anchor body 412 to a bottom surface 416 of the lead anchor body 412 that is opposite the top surface 414. For example, one or more suture holes 408 can be disposed on both a first portion 412a and a second portion 412b of the lead anchor body and extend from a top surface 414 to a bottom surface 416 of the lead anchor body 412. The suture holes 408 are disposed on the lead anchor body 412 such that when the first portion of the lead anchor body 412a is folded over the second portion of the lead anchor body 412b, the one or more suture holes 408 on the first portion of the lead body 412a overlap or align with the one or more suture holes 408 on the second portion of the lead body 412b.

In some embodiments, one or more suture holes 408 are disposed on one or more suture tabs 420. For example, a suture tab 420 can be coupled to and extend away from a surface of the lead anchor body 412. Suture tabs 420 can be coupled to the first portion 412a of the lead anchor body or the second portion 412b of the lead anchor body 412b. Optionally, suture tabs on the two portions 412a, 412b may overlap or align so that a suture can be inserted through the tabs. In some embodiments, suture tabs may not overlap or align and then the suture only goes through a tab coupled to one portion of the lead anchor body.

A method of using the lead anchor 400 to anchor a lead includes fitting (e.g., press fitting) a portion of a lead into the lead channel 402. The first portion of the lead anchor body 412a is then disposed over the second portion of the lead anchor body 412b, for example by folding the first portion 412a over the second portion 412b along an axis of symmetry 410, such that the lead anchor body 412 is in a closed position. In at least some embodiments, the lead anchor is mechanically secured in the second, closed position. For example, the lead anchor 400 may include one or more locking tabs 228 and one or more locking receptacles 230 or some other locking mechanism that secures the lead anchor in the second, closed position. Optionally, the lead anchor 400 is sutured or otherwise secured lead anchor to tissue of a patient, for example, the patient fascia or a ligament.

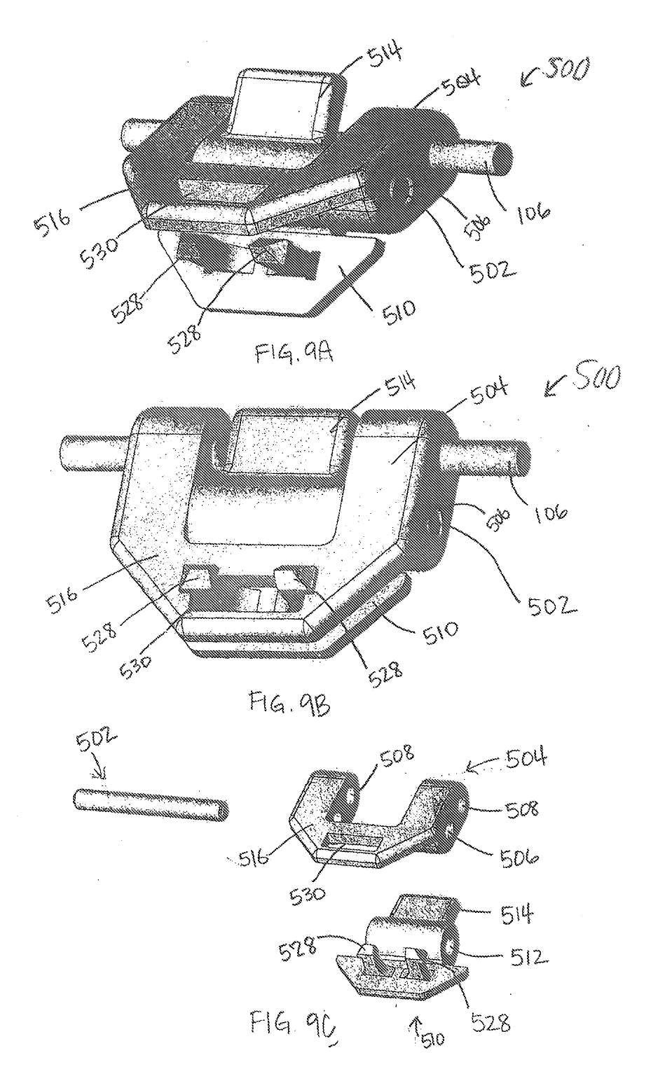

Turning to FIGS. 9A-9E, a lead anchor 500 includes a lead anchor housing 504, a pin 502 and a lead anchor lever 510. The lead anchor housing 504 can be made of any biocompatible material including, for example, silicone, polyurethane, polyetheretherketone (“PEEK”), and the like or combinations thereof. The lead anchor housing 504 includes one or more housing pin openings 506 to receive a portion of the pin 502. The lead anchor housing 504 includes one or more housing lead openings 508 to receive a portion of a lead body 106.

The lead anchor housing 504 includes a projecting edge 516 that extends beyond a portion of the lead anchor housing 504 which includes one or more housing pin openings 506, one or more housing lead openings 508, or both one or more housing pin openings 506 and one or more housing lead openings 508. One or more locking receptacles 530 (FIGS. 9A-9C), one or more locking barbs or tabs (described below), or a combination thereof are disposed on the projecting edge 516 of the lead anchor housing 504.

The pin 502 can be made of any suitable biocompatible material including, for example, rigid plastics or metals such as 316L stainless steel, MP35N (a nickel-cobalt based alloy), titanium alloys such as Ti-6Al-4Vd, and the like or combinations thereof. In some embodiments, the lead anchor housing 504 or the lead anchor lever 510 includes a pin 502 that is not a separate component of the lead anchor 500. For example, the lead anchor housing 504 can include an integrally formed pin 502 and one or more lever pin openings 512 of the lead anchor lever 510 can include a slot that allows the one or more lever pin openings 512 to be snapped on the pin 502 that is integrally formed with the lead anchor housing 504. As an alternative, the lead anchor lever 510 can include an integrally formed pin 502 and one or more housing pin openings 506 can include a slot that allows the one or more housing pin openings 506 to be snapped on the pin that is integrally formed with the lead anchor lever 510.

The lead anchor lever 510 can be made of any biocompatible material including, for example, silicone, polyurethane, polyetheretherketone (“PEEK”), and the like or combinations thereof. The lead anchor lever 510 can include one or more lever pin openings 512 to receive a portion of the pin 502. The one or more lever pin openings 512 and the one or more housing pin openings 506 can be aligned to form a channel to receive a portion of the pin 502. The pin 502 can be placed through the aligned lever pin openings 512 and housing pin openings 506 such that the lead anchor housing 504 and the lead anchor lever 510 are coupled together and able to rotate with respect to one another.

The lead anchor lever 510 can include one or more lead anchor locking barbs 528. The one or more lead anchor locking barbs 528 of a lead anchor lever 510 can engage one or more lead anchor locking receptacles 530 disposed on a lead anchor housing 504 to lock the lead anchor lever to the lead anchor housing. It will be recognized that alternatively the locking barbs may be disposed on the lead anchor housing and the locking receptacles may be disposed on the lead anchor lever.

The lead anchor lever 510 may also include a lead lock tab 514 to contact the lead body 106 disposed in one or more housing lead openings 508 to prevent or reduce migration of the lead body 106 through the lead anchor.

In some embodiments, the lead anchor locking barbs 528 may be designed to pierce, cut or penetrate tissue of a patient. For example, the lead anchor locking barbs 528 may includes a sharp or serrated edge that is capable of piercing, cutting or penetrating a tissue of a patient. For example, the lead anchor locking barbs 528 may be used to penetrate a tissue of a patient and then to engage one or more lead anchor locking receptacles 530. This particular arrangement facilitates locking the lead anchor and attaching the lead anchor to adjacent tissue.

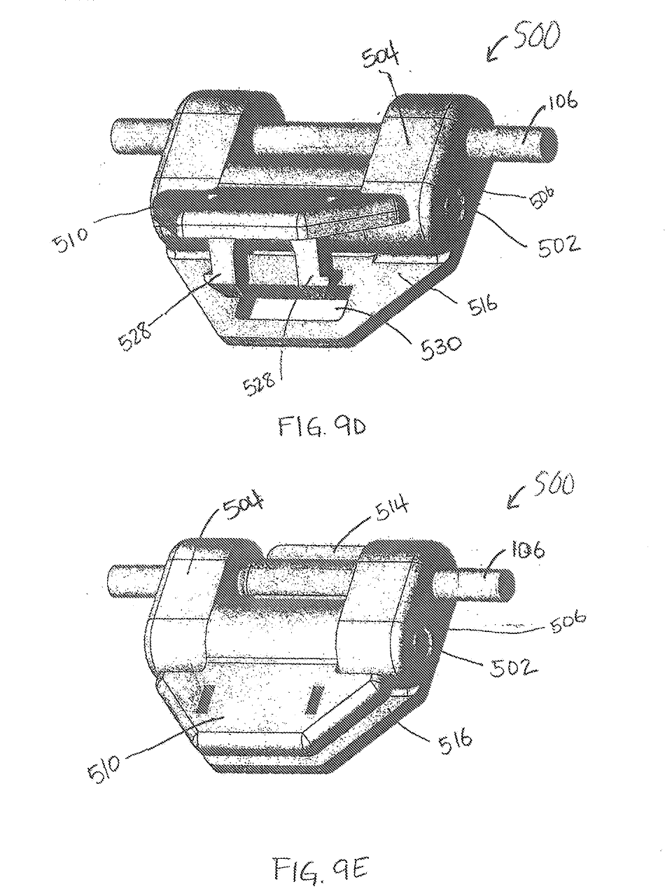

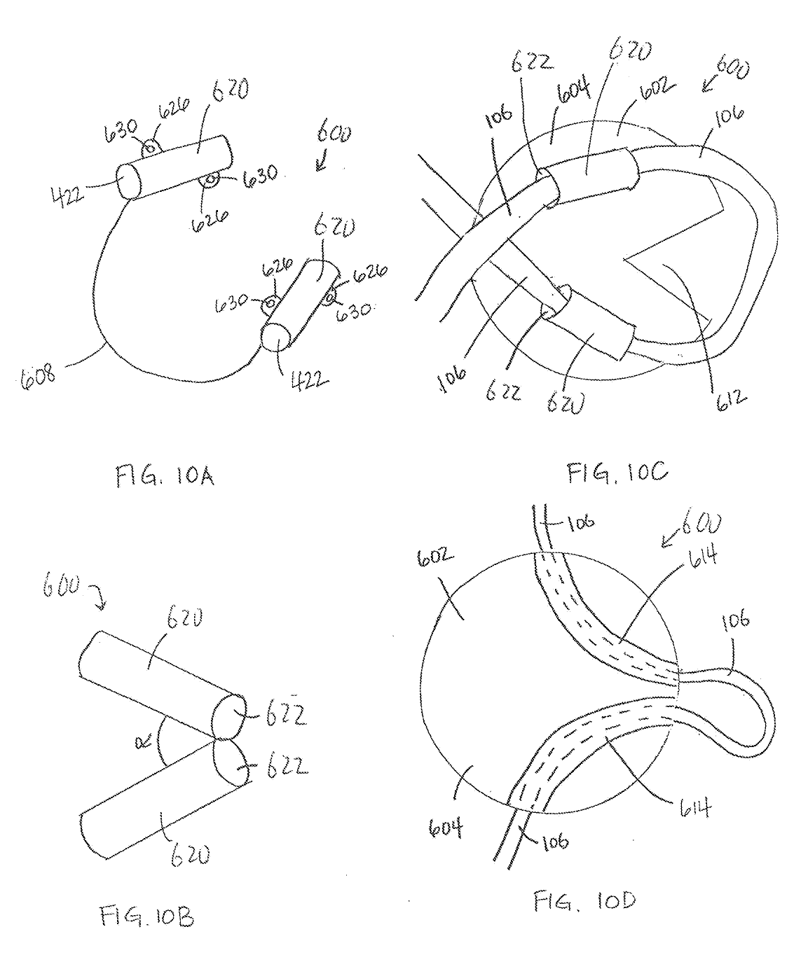

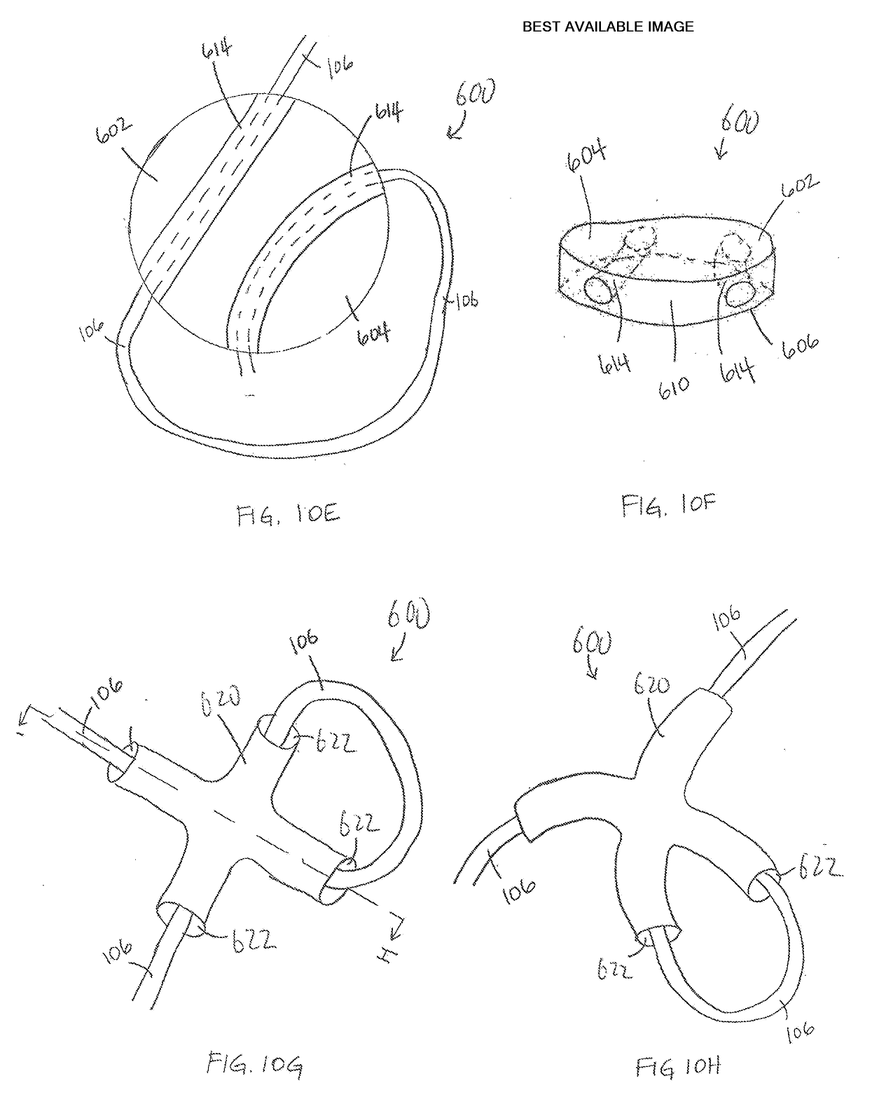

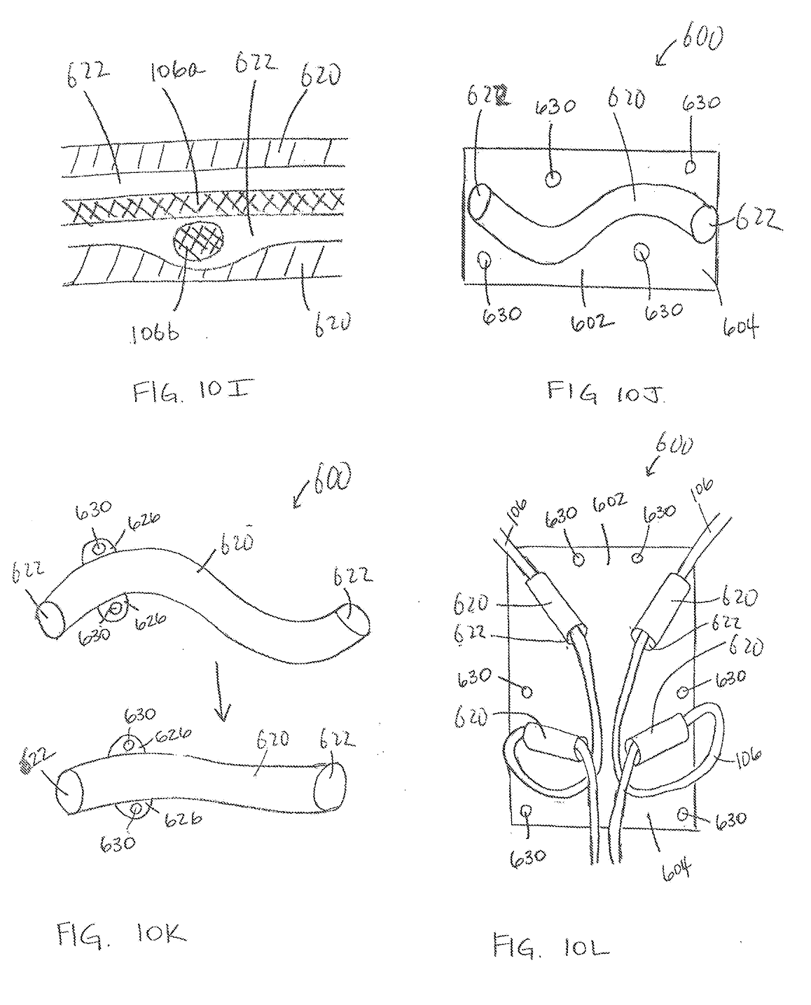

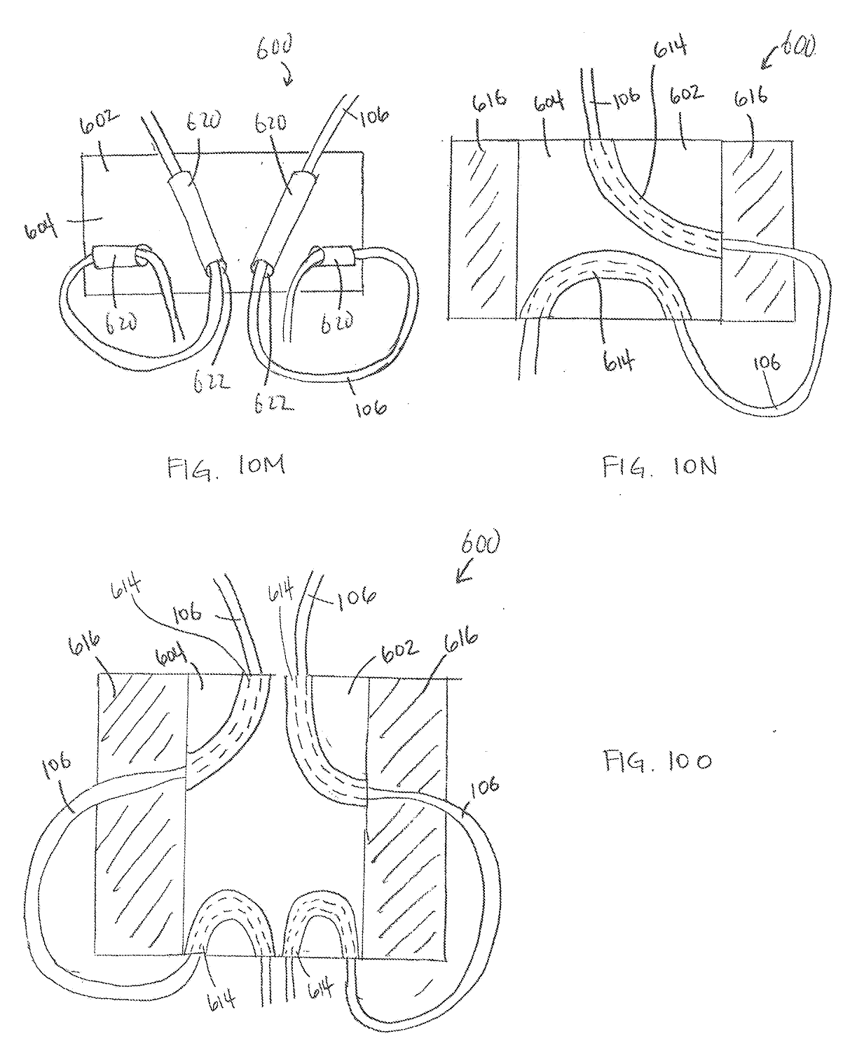

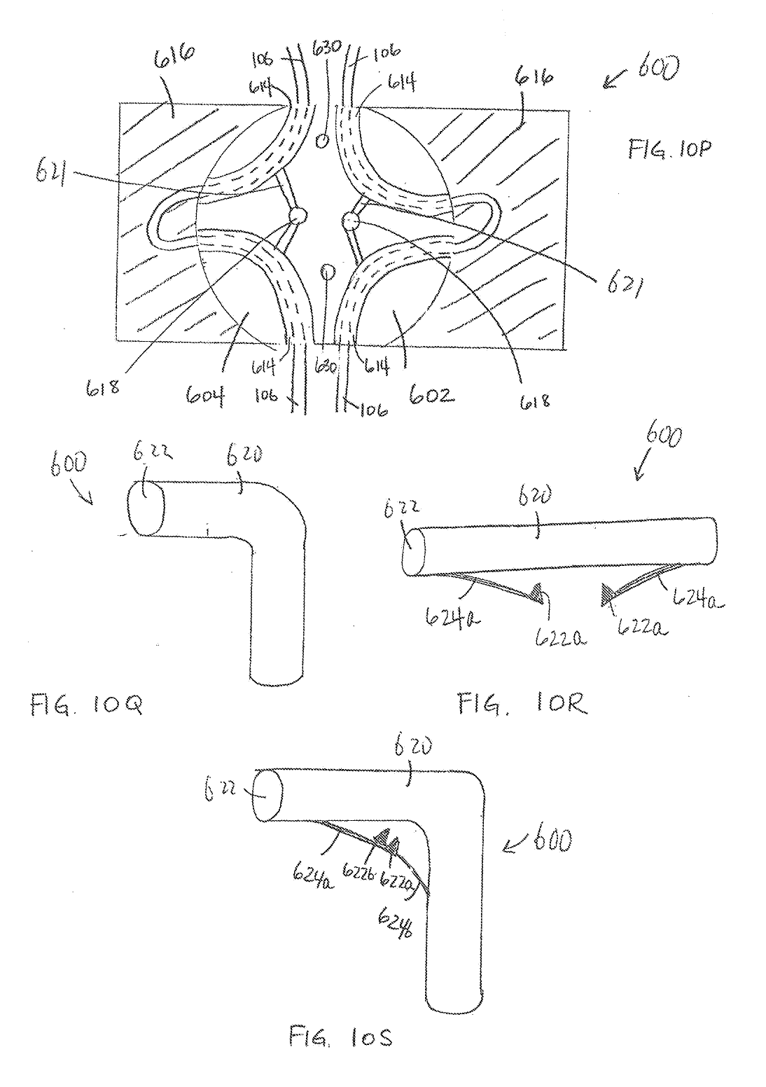

Turning to FIG. 10A, in some embodiments a suture sleeve 600 includes two or more tubular bodies 620 that are coupled together by one or more coupling members 608. A tubular body 620 can be made of any biocompatible material including, for example, silicone, polyurethane, polyetheretherketone (“PEEK”), and the like or combinations thereof. The tubular body 620 may optionally include a single member as illustrated in FIG. 10A such that it is a unitary structure. In some embodiments, the tubular body includes a first portion and a second portion that may be coupled to form a tubular body. In some embodiments, one of the first portion or the second portion includes one or more locking tabs and the other of the first portion or the second portion includes one or more corresponding locking receptacles that can be used to couple the first portion and the second portion of the tubular body.

Similar to the embodiments of FIGS. 12A-12D, in some embodiments a tubular body 620 defines at least one lumen 622 extending through the length of the tubular body 620 to receive at least a portion of at least one lead. For example, in some embodiments, a tubular body 620 defines a single lumen 622. In some embodiments, a tubular body 620 defines two or more lumens 622. In some embodiments, two or more tubular bodies 620 are coupled together or otherwise attached (see, e.g., FIGS. 10G and 10H) and form two or more lumens 622 that may or may not intersect. For example, two or more intersecting tubular bodies 620 can form a cross shape as illustrated in FIGS. 10G and 10H. In some embodiments, two or more tubular bodies 620 can intersect such that a longitudinal axis of a lumen 622 of a first tubular body 620 intersects a longitudinal axis of a lumen 622 of a second tubular body 620. The longitudinal axes of the lumen 622 of the first tubular body 620 and the lumen 622 of the second tubular body 620 may be in the same horizontal plane such that the lumens 622 intersect or may be in different horizontal planes such that the lumens 622 do not intersect.

In some embodiments, at least one tubular body 620 defines a lumen 622 that is straight such that a portion of a lead disposed within the lumen follows a straight path as illustrated in, for example, FIGS. 10C and 10M. In some embodiments, at least one tubular body 620 defines a lumen 622 that is curved such that a portion of a lead disposed within the lumen 622 follows a curved path. In some embodiments, at least one tubular body 620 defines a lumen 622 that is S-shaped as illustrated in FIG. 10J. In some embodiments, two or more lumens 622 defined by a tubular body 620 are parallel. In some embodiments, two or more lumens 622 defined by a tubular body 620 are perpendicular and may or may not intersect. For example, the tubular body 620 illustrated in FIG. 10G may define two lumens 622 that are perpendicular but do not intersect because a first lumen 622 lies above or below a second lumen 622. Alternatively, the tubular body 620 illustrated in FIG. 10G may define two lumens 622 that are perpendicular and intersect as illustrated in FIG. 10I, which is a cross-sectional view of a portion of the tubular body 620 of FIG. 10G along line I-I.

In some embodiments, one or more suture tabs 626 are coupled to and extend away from a surface of the tubular body 620 and include one or more suture holes 630 as in FIG. 10A. Suture tabs 626 may be disposed anywhere along the surface of the tubular body 620. For example, one or more suture tabs 626 may be disposed on only one end of a tubular body 620 as illustrated in FIG. 10K, or on both ends of a tubular body 620.

The coupling member 608 can be made of any biocompatible material. In some embodiments, the coupling member 608 is made of a memory metal such as, for example, nitinol that allows the tubular bodies 620 connected by the coupling member 608 to be manipulated during implantation and then resume their original orientation with respect to each other.

Turning to FIGS. 10A and 10B, in some embodiments, two or more tubular bodies 620 are coupled together with a coupling member 608 that maintains the tubular bodies 620 in the same orientation with respect to one another. A coupling member 608 may have any shape and may include a plate, rod, V-shaped member, mesh or the like. For example, a first end of a V-shaped coupling member 608 can optionally be coupled to a first tubular body 620 and a second end of a V-shaped coupling member 608 can optionally be coupled to a second tubular body 620.

Turning to FIGS. 10C-10O, in some embodiments, a suture sleeve 600 includes a suture sleeve base 602. The suture sleeve base 602 may be made of any biocompatible material including, for example, silicone, polyurethane, polyetheretherketone (“PEEK”), and the like or combinations thereof. In some embodiments, the suture sleeve base 602 includes a mesh structure (not shown) to add strength to the suture sleeve base 602 without adding stiffness. In some embodiments, the suture sleeve base 602 is made entirely of a mesh structure (e.g., hernia mesh). In some embodiments, the mesh structure allows tissue in-growth, which may act to further anchor the suture sleeve 600 to tissue of a patient.

The suture sleeve base 602 includes a top surface 604, a bottom surface 606 (see FIG. 10F) that is opposite the top surface, and at least one side 610 (see FIG. 10F) connecting the top surface 604 and the bottom surface 606. The top surface 604 and the bottom surface 606 can have any shape including, for example, a regular or irregular circular (see, e.g., FIGS. 10C-10F), semi-circular, square, triangular or rectangular (see, e.g., FIGS. 10J, 10L-10O) shape. For example, the suture sleeve base 602 may optionally have the shape of a circular or semi-circular disc.

In some embodiments, an adhesive (not shown) may be disposed on at least a portion of a surface of the suture sleeve base 602. For example, adhesive may be disposed on at least a portion of the bottom surface 606 of the suture sleeve base 602. In some embodiments, the adhesive is used to aid in holding the suture sleeve in place while the lead is being disposed in the suture sleeve and/or while the suture sleeve is being sutured to surrounding tissue. In some embodiments, a protective backing (not shown) is disposed on at least a portion of the adhesive. In some embodiments, a protective backing is disposed completely over the adhesive. In some embodiments, a suture sleeve 600 is packaged with a protective backing disposed at least partially over the adhesive and the protective backing is removed prior to implanting the suture sleeve 600.

In some embodiments, the suture sleeve base 602 includes a cut-out 612. In one embodiment, a cut-out 612 is illustrated in FIG. 10C. The cut-out 612 may have any shape including, for example, a regular or irregular circular, semi-circular, rectangular, square or triangular shape. In some embodiments, the cut-out 612 extends from an edge of the suture sleeve base 602 towards a center of the suture sleeve base 602. In some embodiments, a cut-out 612 allows two or more tubular bodies 620 disposed on the suture sleeve base 602 to flex toward and away from each other. Such flexing has many advantages including, for example, allowing for increased strain relief and force redirection.

In some embodiments, one or more tubular bodies 620 are disposed on the suture sleeve base 602 as illustrated in FIGS. 10C, 10L and 10M. For example, one or more tubular bodies 620 may optionally be disposed on a top surface 604 of the suture sleeve base 602. In some embodiments, two or more tubular bodies 620 are disposed on a suture sleeve base 602 such that a longitudinal axis of a first tubular body 620 is parallel to a longitudinal axis of a second tubular body 620. In some embodiments, two or more tubular bodies 620 are disposed on a suture sleeve base 602 such that longitudinal axis of a first tubular body 620 is perpendicular to a longitudinal axis of a second tubular body 620. In some embodiments, two or more tubular bodies 620 are disposed on a suture sleeve base 602 such that a longitudinal axis of a first tubular body 620 forms an angle with the longitudinal axis of a second tubular body 620, wherein the angle is from 15°-75°. For example, an angled formed by the longitudinal axes of a first tubular body 620 and second tubular body 620 can be at least 15°, 30°, 35°, 50°, 55°, 60°, 65°, 70° or 75°.

Turning to FIGS. 10D-10F, in some embodiments, the suture sleeve base 602 includes one or more suture sleeve base lumens 614 to receive at least a portion of a lead. A suture sleeve base lumen 614 may be straight, such that a portion of a lead disposed within the suture sleeve base lumen 614 follows a straight path, or curved, such that a portion of a lead disposed within the suture sleeve base lumen 614 follows a curved path, such as an “S” shaped path. A suture sleeve base lumen 614 may begin and end on any side of the suture sleeve base. For example, in some embodiments, a suture sleeve base lumen 614 begins on a first side of a suture sleeve base 602 and exits on a second side of the suture sleeve base 602 that is opposite the first side. In some embodiments, a suture sleeve base lumen 614 begins and exits on the same side of a suture sleeve base 602. In some embodiments, two or more suture sleeve base lumens 614 promote bowing (see, e.g., FIG. 10D) or looping (see, e.g., FIG. 10E) of the lead.

In some embodiments, two or more suture sleeve base lumens 614 are parallel to one another. In some embodiments, two or more suture sleeve base lumens 614 are perpendicular to one another and may or may not intersect. For example, in some embodiments, a first suture sleeve base lumen 614 is perpendicular to a second suture sleeve base lumen 614, but does not intersect the second suture sleeve base lumen 614 because it is disposed either above or below the second suture sleeve base lumen 614.

The inner diameter of a lumen 622 of a tubular body 620 or a suture sleeve base lumen 614 may have any size as long as it can receive a portion of a lead. In some embodiments, the surface of a lumen 622 or a suture sleeve base lumen 614 includes flanges, protrusions or bumps that prevent or reduce migration of the lead within the lumen 622 or suture sleeve base lumen 614. In some embodiments, a portion of a lead is secured in the lumen 622 or suture sleeve base lumen 614 by pressure from an external force such as a suture. In some embodiments, a portion of a lead is secured in the lumen 622 or suture sleeve base lumen 614 using adhesive. For example, a suture sleeve base 602 may include one or more adhesive injection ports 618 (see FIG. 10P) that are either directly coupled to one or more suture sleeve base lumens 614 or that are coupled to a suture sleeve base lumen 614 by one or more adhesive tunnels 621. Likewise, a tubular body 620 may include one or more adhesive injections ports 618 that can be used to introduce adhesive into the lumen 622. In some embodiments, a portion of a tubular body 620 or a suture sleeve base 602 that flanks a lumen 622 or a suture sleeve base lumen 614, respectively, can include an expanding material 670.

A suture sleeve base 602 may include one or more suture sleeve suture holes 630 that can, for example, extend from a top surface 604 of the suture sleeve base 602 through the suture sleeve base 602 to the bottom surface 606 of the suture sleeve base 602. The suture holes 630 can be used to secure the suture sleeve base 602 to tissue of a patient, for example to patient fascia or a ligament. In some embodiments, one or more sutures may be pre-placed in one or more suture holes 630. In some embodiments, one or more sutures may be pre-wrapped (“laced”) around a suture sleeve 600.

In some embodiments, at least a portion of a tubular body 620 or a suture sleeve base 602 includes a porous material 616 (see, e.g., FIGS. 10N, 10O, and 10P). The porous material 616 may enhance the in-growth of tissue, which may aid anchoring of the device over time. In some embodiments, a substance that promotes tissue growth such as, for example, a growth hormone or a growth factor or another material such as expanded polytetrafluorothylene (PTFE) is integrated into the device, or coated on the device, to aid in tissue in-growth.

Turning to FIG. 10Q, in some embodiments, a suture sleeve 600 maintains a same shape. For example, in some embodiments, a suture sleeve 600 includes a tubular body 620 that is permanently bent such that the tubular body 620 forms an angle in the range of 10° to 170° (for example, a 10 °, 60°, 30°, 60°, 50°, 60°, 70°, 60°, 100°, 110°, 120°, 130°, 140°, 150°, 160°, or 170° angle.) In some embodiments, a tubular body 620 includes two or more locking connectors 624 and two or more locking teeth 622. For example, in FIG. 10R, the tubular body 620 includes a first locking tooth connector 624a that is coupled to a first end of the tubular body 620 and to a first locking tooth 622a. The tubular body 620 illustrated in FIG. 10R also includes a second locking tooth connector 624b that is coupled to a second end of the tubular body 620 and to a second locking tooth 622b. In some embodiments, the tubular body 620 can be folded and locking teeth 622a and 622b can interlock as illustrated in FIG. 10S. For example, the locking teeth 622 can be similar to those on a hemostat. As will be recognized, any number of locking teeth 622 can be disposed on a locking tooth connector 624. As will also be recognized, two or more locking teeth connectors 624 can be disposed anywhere along the surface of a tubular body 620 to achieve the desired tubular body 620 shape when the locking teeth are interlocked.

The suture sleeves 600 described above have several advantages including providing strain relief (e.g., absorbing, eliminating or reducing lead movement distal to the anchoring device as a result of proximal axial force) and force redirection (e.g., changing direction of a force to prevent or minimize lead slippage through the anchoring device, for example, by changing the force from a pulling force to a bending force). For example, a S-shaped tubular body 620 may aid strain relief because as the tubular body 620 is pulled (e.g., from posture changes of a patient), the S may straighten out, thereby absorbing some of the strain and minimizing lead movement beyond the tubular body 620, as illustrated in FIG. 10K.

Suture sleeves 600 that include multiple tubular members 620 or suture sleeve base lumens 614 in pre-set arrangements may be especially useful, for example, in instances where specific suture sleeve orientations and lead configurations are required. In this situation, a suture sleeve 600 that includes multiple tubular members 620 or suture sleeve base lumens 614 in a pre-set arrangement may eliminate many steps in the surgical procedure and reduce the number of suture sleeves 600 needed to one or a few. The suture sleeves 600 described herein may receive one or more portions of one or more leads.

One example of a method of implanting a lead includes disposing a first portion of a lead, such as a first portion of a lead body, into a first lumen 622 of a first tubular body 620. A second portion of a lead is disposed through a second lumen 622 of the same or a different tubular body. Optionally, a portion of the lead body is looped or bowed prior to insertion into the second lumen. The portion of the lead body may be bowed or looped to increase, for example, strain relief and force redirection.

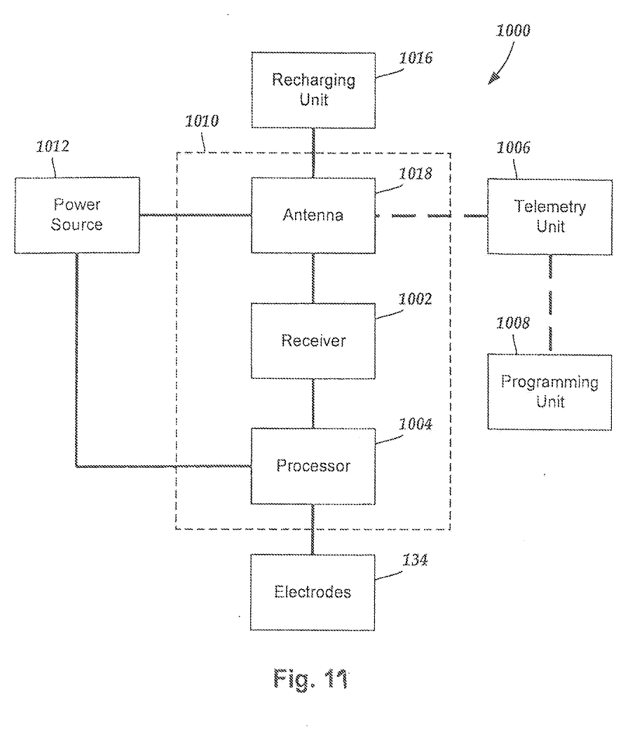

FIG. 11 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

1.-40. (canceled)

41. A suture sleeve comprising:
a plurality of tubular bodies, wherein each tubular body defines at least one lumen that is configured and arranged to receive a portion of a lead;
a coupling member that couples the plurality of tubular bodies and that is configured and arranged to that allow the tubular bodies to be manipulated during implantation and then resume their original orientation with respect to each other.

42. The suture sleeve of claim 41, wherein the coupling member comprises nitinol.

43. The suture sleeve of claim 41, wherein one or. more of the tubular bodies comprises one or more suture tabs and one or more suture holes disposed on each suture tab.

44. The suture sleeve of claim 41, wherein the at least one lumen is non-linear.

45. A suture sleeve comprising:
a suture sleeve base that comprises a top surface, a bottom surface and at least one side surface that couples the top surface and the bottom surface; and
one or more tubular bodies disposed on the suture sleeve base in a pre-determined arrangement, wherein each tubular body defines at least one lumen extending through the length of the tubular body and wherein each lumen is configured and arranged to receive at least a portion of a lead.

46. The suture sleeve of claim 45, wherein the suture sleeve base comprises a cut-out that extends from an edge of the suture sleeve base toward a center of the suture sleeve base.

47. The suture sleeve of claim 45, wherein the suture sleeve base comprises at least one suture hole that extends from the top surface of the suture sleeve base through the suture sleeve base to the bottom surface of the suture sleeve base.

48. The suture sleeve of claim 45, further comprising a porous material.

49. The suture sleeve of claim 48, wherein the porous material comprises a growth hormone or growth factor.

50-58. (canceled)

59. A suture sleeve comprising:
two or more tubular bodies, wherein each tubular body defines at least one lumen extending through the length of the tubular body, wherein the at least one lumen is configured and arranged to receive a portion of a lead, and wherein the two or more tubular bodies intersect to form a cross shape.

60. The suture sleeve of claim 59, wherein the lumen of the first tubular body intersects the lumen of the second tubular body.

61. The suture sleeve of claim 59, wherein the lumen of the first tubular body does not intersect the lumen of the second tubular body.

62. The suture sleeve of claim 59, wherein at least one lumen is curved.
